(19) United States
(12) Patent Application Publication
(10) Pub. No.: US 2023/0263956 A1
(43) Pub. Date: Aug. 24, 2023

(54) INGESTIBLE DRUG DELIVERY DEVICE

(71) Applicants: Massachusetts Institute of Technology, Cambridge, MA (US); Novo Nordisk A/S, Bagsværd (DK)

(72) Inventors: Graham Arrick, San Francisco, CA (US); Declan Gwynne, Everett, MA (US); Jacob Wainer, Malden, MA (US); Carlo Giovanni Traverso, Newton, MA (US); Drago Sticker, Malmo (SE); Cody Cleveland, København V (DK); Aghiad Ghazal, Bagsværd (DK); Jorrit Jeroen Water, Frederiksberg (DK); Brian Mouridsen, Bagsværd (DK); Mette Poulsen, Bagsværd (DK); Dan Nørtoft Sørensen, Allerød (DK); Jacob Pyung Hwa Jepsen, Frederiksberg (DK)

(73) Assignees: Massachusetts Institute of Technology, Cambridge, MA (US); Novo Nordisk A/S, Bagsværd (DK)

(21) Appl. No.: 18/020,386

(22) PCT Filed: Aug. 9, 2021
(86) PCT No.: PCT/US2021/045196

§ 371 Date: Feb. 8, 2023

Related U.S. Application Data
(60) Provisional application No. 63/063,818, filed on Aug. 10, 2020.

(51) Int. Cl. A61M 5/20; A61M 5/30

(52) U.S. Cl. CPC A61M 5/20; A61M 5/30

(57) ABSTRACT

A drug delivery device for administration to a subject may include a reservoir containing an active pharmaceutical ingredient and a potential energy source. The drug delivery device may also include a trigger operatively associated with the potential energy source. The trigger may be configured to actuate at a predetermined location within the subject to deploy a jet of the active pharmaceutical ingredient into a tissue of an adjacent portion of the gastrointestinal tract. In some instances, the jet may be deployed into tissue of the stomach and/or small intestine of the subject. Further, in some embodiments, the operating parameters of the jet may be selected such that the jet penetrates the tissue of the gastrointestinal tract to form a depot of the active pharmaceutical ingredient disposed within the tissue.

### CROSS-REFERENCE TO RELATED APPLICATIONS

This application claims the benefit under 35 U.S.C. § 119(e) of U.S. Provisional Application Ser. No. 63/063,818, filed on Aug. 10, 2020, which is incorporated herein in its entirety.

### FIELD

Disclosed embodiments are related to ingestible drug delivery devices and related methods of use.

### BACKGROUND

Certain therapeutics are composed of large and complex molecules that denature readily when administered via the oral-gastrointestinal (GI) route. Accordingly, patients who need these therapeutics typically use more invasive forms of drug administration that are outside the GI route including, for example, subcutaneous injection.

### SUMMARY

In some embodiments, a drug delivery device configured for administration to a subject includes a reservoir configured to contain an active pharmaceutical ingredient, a potential energy source; a trigger operatively associated with the potential energy source, where the trigger is configured to actuate within a stomach of the subject, and an outlet in fluid communication with the reservoir. When the trigger is actuated the potential energy source compresses the reservoir to jet the active pharmaceutical ingredient from the reservoir through the outlet with a velocity sufficient to penetrate a tissue of the stomach adjacent to the outlet. A peak power provided by the potential energy source to form the jet of the active pharmaceutical ingredient is between 9 Watts (W) and 130 W.

In some embodiments, a drug delivery device configured for administration to a subject includes a reservoir configured to contain an active pharmaceutical ingredient, a potential energy source, a trigger operatively associated with the potential energy source, where the trigger is configured to actuate within a stomach of the subject, and an outlet in fluid communication with the reservoir. When the trigger is actuated the potential energy source compresses the reservoir to jet the active pharmaceutical ingredient from the reservoir through the outlet with a velocity sufficient to penetrate a tissue of the stomach adjacent to the outlet. The outlet, the reservoir, and the potential energy source are configured to form a depot of the active pharmaceutical ingredient in a tissue of the stomach without perforating a muscularis layer of the stomach.

In some embodiments, a method of administering an active pharmaceutical ingredient to a subject includes triggering deployment of a jet of the active pharmaceutical ingredient within a stomach of the subject and penetrating a tissue of the stomach of the subject with the jet, where a peak power applied to form the jet of the active pharmaceutical ingredient is between 9 Watts (W) and 130 W.

In some embodiments, a method of administering an active pharmaceutical ingredient to a subject includes triggering deployment of a jet of the active pharmaceutical ingredient within a stomach of the subject, penetrating a tissue of the stomach of the subject with the jet, and forming a depot of the active pharmaceutical ingredient within the tissue of the stomach without perforating a muscularis layer of the stomach.

In some embodiments, a drug delivery device configured for administration to a subject includes a reservoir configured to contain an active pharmaceutical ingredient, a potential energy source, a trigger operatively associated with the potential energy source, where the trigger is configured to actuate within a small intestine of the subject, and an outlet in fluid communication with the reservoir. When the trigger is actuated, the potential energy source compresses the reservoir to jet the active pharmaceutical ingredient from the reservoir through the outlet with a velocity sufficient to penetrate a tissue of the small intestine adjacent to the outlet. A peak power provided by the potential energy source to form the jet of the active pharmaceutical ingredient is between 3 Watts (W) and 6.5 W.

In some embodiments, a drug delivery device configured for administration to a subject includes a reservoir configured to contain an active pharmaceutical ingredient, a potential energy source, a trigger operatively associated with the potential energy source, where the trigger is configured to actuate within a small intestine of the subject, and an outlet in fluid communication with the reservoir. When the trigger is actuated the potential energy source compresses the reservoir to jet the active pharmaceutical ingredient from the reservoir through the outlet with a velocity sufficient to penetrate a tissue of the small intestine adjacent to the outlet. The outlet, the reservoir, and the potential energy source are configured to form a depot of the active pharmaceutical ingredient in a tissue of the small intestine without perforating a muscularis layer of the small intestine.

In some embodiments, a method of administering an active pharmaceutical ingredient to a subject includes triggering deployment of a jet of the active pharmaceutical ingredient within a small intestine of the subject and penetrating a tissue of the small intestine of the subject with the jet, where a peak power applied to form the jet of the active pharmaceutical ingredient is between 3 Watts (W) and 6.5 W.

In some embodiments, a method of administering an active pharmaceutical ingredient to a subject includes triggering deployment of a jet of the active pharmaceutical ingredient within a small intestine of the subject, penetrating a tissue of the small intestine of the subject with the jet, and forming a depot of the active pharmaceutical ingredient within the tissue of the small intestine without perforating a muscularis layer of the small intestine.

In some embodiments, a drug delivery device configured for administration to a subject includes a reservoir configured to contain an active pharmaceutical ingredient, a potential energy source, a trigger operatively associated with the potential energy source, wherein the trigger is configured to actuate in response to one or more predetermined conditions, and an outlet in fluid communication with the reservoir. In some embodiments, when the trigger is actuated the potential energy source compresses the reservoir to jet the active pharmaceutical ingredient from the reservoir through the outlet with a velocity between 20 m/s and 250 m/s. In some embodiments, a peak power provided by the potential energy source to form the jet of the active pharmaceutical ingredient is between 9 Watts (W) and 130 W. In some embodiments, the trigger may be configured to actuate within a stomach of the subject. In some embodiments, when the trigger is actuated the potential energy source may compress the reservoir to jet the active pharmaceutical ingredient from the reservoir through the outlet with a velocity sufficient to penetrate a tissue of the stomach adjacent to the outlet.

In some embodiments, a drug delivery device configured for administration to a subject includes a reservoir configured to contain an active pharmaceutical ingredient, a potential energy source, a trigger operatively associated with the potential energy source, wherein the trigger is configured to actuate in response to one of more predetermined conditions, and an outlet in fluid communication with the reservoir. In some embodiments when the trigger is actuated the potential energy source compresses the reservoir to jet the active pharmaceutical ingredient from the reservoir through the outlet with a velocity between 20 m/s and 250 m/s. In some embodiments, the outlet, the reservoir, and the potential energy source are configured to form a depot of the active pharmaceutical ingredient in a tissue of the stomach without perforating a muscularis layer of the stomach. In some embodiments, the trigger may be configured to actuate within a stomach of the subject. In some embodiments, when the trigger is actuated the potential energy source may compress the reservoir to jet the active pharmaceutical ingredient from the reservoir through the outlet with a velocity sufficient to penetrate a tissue of the stomach adjacent to the outlet.

In some embodiments, a drug delivery device configured for administration to a subject includes a reservoir configured to contain an active pharmaceutical ingredient, a potential energy source, a trigger operatively associated with the potential energy source, wherein the trigger is configured to actuate in response to one or more predetermined conditions, and an outlet in fluid communication with the reservoir. In some embodiments, when the trigger is actuated the potential energy source compresses the reservoir to jet the active pharmaceutical ingredient from the reservoir through the outlet with a velocity between 40 m/s and 80 m/s. In some embodiments, a peak power provided by the potential energy source to form the jet of the active pharmaceutical ingredient is between 3 W and 6.5 W. In some embodiments, the trigger may be configured to actuate within a small intestine of the subject. In some embodiments, when the trigger is actuated the potential energy source compresses the reservoir to jet the active pharmaceutical ingredient from the reservoir through the outlet with a velocity sufficient to penetrate a tissue of the small intestine adjacent to the outlet.

In some embodiments, a drug delivery device configured for administration to subject includes a reservoir configured to contain an active pharmaceutical ingredient, a potential energy source, a trigger operatively associated with the potential energy source, wherein the trigger is configured to actuate in response to one or more predetermined conditions, and an outlet in fluid communication with the reservoir. In some embodiments, when the trigger is actuated the potential energy source compresses the reservoir to jet the active pharmaceutical ingredient from the reservoir through the outlet with a velocity between 40 m/s and 80 m/s adjacent to the outlet. In some embodiments, the outlet, the reservoir, and the potential energy source are configured to form a depot of the active pharmaceutical ingredient in a tissue of the small intestine without perforating a muscularis layer of the small intestine. In some embodiments, the trigger may be configured to actuate within a small intestine of the subject. In some embodiments, when the trigger is actuated the potential energy source compresses the reservoir to jet the active pharmaceutical ingredient from the reservoir through the outlet with a velocity sufficient to penetrate a tissue of the small intestine adjacent to the outlet.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A depicts a schematic of one embodiment of a drug delivery device;

FIG. 1B depicts a cross-sectional view of the drug delivery device of FIG. 1A in a first state;

FIG. 1C depicts a cross-sectional view of the drug delivery device of FIG. 1A in a second state;

FIG. 2A depicts one embodiment of a drug delivery device in a first state;

FIG. 2B depicts a cross-sectional view of the drug delivery device of FIG. 2A in a second state;

FIG. 3 depicts one embodiment of a drug delivery device passing through the gastrointestinal system of a subject;

FIG. 4 depicts a schematic graph of jet power versus time;

FIG. 5A depicts a graph of calculated jet force versus time for different nozzle sizes;

FIG. 5B depicts a graph of calculated jet power versus time for different nozzle sizes;

FIG. 5C depicts a graph of experimental jet force versus time for different nozzle sizes;

FIG. 5D depicts a graph of experimental jetting power versus jet diameter;

FIG. 5E depicts a graph of experimental delivery efficiency versus nozzle size;

FIG. 5F depicts a graph of experimental jetting power versus nozzle size;

FIG. 6A depicts measured and predicted jet performance parameters in different anatomical structures along the gastrointestinal tract;

FIG. 6B depicts a graph of measured jet injection efficiency versus jetting force for a variety of gastrointestinal tract tissues;

FIG. 7 is a schematic diagram of a tethered drug delivery device administering an API within a stomach;

FIG. 8A is a preliminary experimental summary of parametric inputs including jetting force and their resulting delivery efficiencies in stomach tissue;

FIG. 8B is a preliminary experimental summary of parametric inputs including jetting pressure and their resulting delivery efficiencies in stomach tissue;

FIG. 9A is a preliminary experimental summary of parametric inputs including jetting force and their resulting delivery efficiencies in intestinal tissue; and

FIG. 9B is a preliminary experimental summary of parametric inputs including jetting pressure and their resulting delivery efficiencies in intestinal tissue.

### DETAILED DESCRIPTION

Large and complex molecules that denature readily when administered via the oral-gastrointestinal (GI) route are regularly administered as a part of therapeutic treatments. Patients requiring these therapeutics oftentimes must use more invasive forms of drug administration such as subcutaneous injection. The use of these more invasive forms of delivery sometimes lead to lapses in routine adherence and/or reduced quality of life.

In view of the above, the inventors have recognized the benefits of ingestible delivery devices that leverage needleless micro-jets to deliver a dose of a desired active pharmaceutical ingredient (API) at a desired location along the gastrointestinal (GI) tract without compromising drug-purity, efficacy, and/or dosage. In particular, the inventors have recognized the benefits of an ingestible delivery device employing a trigger that automatically releases a dose at a desired location within the GI tract. As used herein, the GI tract includes the esophagus, the stomach, the duodenum, the jejunum, the small intestine, and the large intestine. The delivery device may be suitable to delivery of large and complex molecules, such as proteins and other biologics, that may otherwise be unsuitable for delivery through the GI tract, though any appropriate API may be used. According to exemplary embodiments described herein, an ingestible delivery device employing micro-jetting for delivery of an active pharmaceutical ingredient (API) has many potential benefits. First, an ingestible delivery device according to exemplary embodiments described herein may not include sharp points. Second, micro-jects obviate the mechanisms associated with actuating and/or retracting a needle, thereby reducing system complexity and cost relative to needle-based systems. The use of jet deployed APIs may also result in significant increases in the bioavailability of the API on par with subcutaneous injections as compared to other ingested API's provided with common chemical permeation enhancers (approximately 2% bioavailability). Lastly, implementation of needle-free delivery systems of exemplary embodiments described herein may result in less pain and/or trauma at the site of injection relative to needle-based delivery, as well as enhanced pharmacokinetics (PK).

In view of the above, the Inventors have recognized the benefits associated with jet-based injection of an active pharmaceutical ingredient into the gastrointestinal tissue of a subject. However, due to the jet-based deployment of the active pharmaceutical ingredient (API) being needle-free, control of the various operating parameters associated with the jet may dictate which anatomical structures the jet of API is deployed into. For example, a drug delivery device may be configured to provide a jet of API that is appropriately tuned for: intraluminal delivery of the API into an intraluminal space of the gastrointestinal tract (i.e. a wet shot); intramucosal delivery of the API into a mucosal tissue of the gastrointestinal tract; intrasubmucosal delivery of the API into a submucosal tissue of the gastrointestinal tract; intraperitoneal delivery of the API into the peritoneal space of the subject; combinations of the foregoing; and/or any other appropriate form of delivery. Depending on the target tissue and the specific parameters of the jet, in some embodiments, a depot of the API may be formed in the target tissue where the depot may be a volume of the API disposed in the target tissue and/or between layers of different tissue of the gastrointestinal tract. In some embodiments, a drug delivery device may be configured for delivering a jet of an API into tissue within a stomach and/or small intestine of a subject. Specific operating parameters which may be selected to optimize a jet for delivery into these different tissue locations may include, for example, jet power, diameter, dosage, standoff distance, fluid viscosity, fluid density, and other appropriate parameters as elaborated on further below.

In some embodiments, an active pharmaceutical ingredient may be administered to a subject by triggering deployment of a jet of the active pharmaceutical ingredient from a drug delivery device when the drug delivery device is located at a desired location within the gastrointestinal tract of a subject. According to exemplary embodiments described herein, a jet may be triggered by a predetermined condition. In some embodiments, the predetermined condition includes one or more of a predetermined time after ingestion of the drug delivery device, a predetermined location in the GI tract, physical contact with the GI tract, physical manipulation in the GI tract (e.g., compression via peristalsis), one or more characteristics of the GI tract (e.g., pH, pressure, acidity, temperature, etc.), or combinations thereof. In some embodiments, the jet may be deployed when the drug delivery devices located in a stomach and/or small intestine of a subject. In either case, the operating parameters of the jet may be appropriately selected such that the jet is emitted from the drug delivery device with sufficient velocity such that the jet penetrates a tissue of the gastrointestinal tract of the subject adjacent to the drug delivery device to form a depot of the active pharmaceutical ingredient within the tissue of the gastrointestinal tract proximate to the drug delivery device upon actuation. In some embodiments, the jet may form the depot of the active pharmaceutical ingredient in the tissue of the gastrointestinal tract without perforating the muscularis layer of the gastrointestinal tract underlying the injection site where the jet impinges on the tissue of the gastrointestinal tract. As used herein, the terms “proximate to” and “adjacent to” are used interchangeably and defined herein to mean the specified elements being in direct contact or being in sufficiently close proximity in space to accomplish a specified function, such as being spaced apart for a standoff distance as used herein

Without wishing to be bound by theory, the Inventors have recognized that one of the controlling parameters for delivering an active pharmaceutical ingredient (API) to a desired target location within the tissue of the gastrointestinal tract of a subject is a peak power of a jet during delivery of the API into the target tissue. For example, a peak power of a jet used to deploy an API into a target tissue may be selected such that the jet forms a depot of the API disposed in the target tissue without perforating underlying layers of the gastrointestinal tract. Advantageously, this parameter may take into account various other operating parameters such as deployment force, density, viscosity, area of the jet, and velocity of the jet enabling the design and comparison of delivery devices with different API's and/or deployment systems for a desired application. Additionally, the Inventors have recognized that a peak power appropriate for forming a depot in a target tissue varies based on a location of a delivery device within a gastrointestinal tract of a subject. For example, an optimal peak power for operation within the stomach of a subject is different from an optimal peak power for operation within a small intestine of a subject and/or other portion of the gastrointestinal tract.

As noted above, in some embodiments, it is desirable to deliver an active pharmaceutical ingredient to a tissue of the stomach of a subject. Accordingly, an appropriate peak power may be selected to allow a jet to penetrate tissue of the stomach proximate to a drug delivery device disposed within a stomach of a subject. In some instances, the peak power may be selected to avoid perforating a muscularis layer of the stomach. In such an embodiment, a peak power of a jet oriented towards a surface of a subject's stomach may be greater than or equal to 9 W, 10 W, 12 W, 15 W, 20 W, 25 W, 50 W, 100 W, and/or any other appropriate power. Correspondingly, a peak power of the jet may be less than or equal to 130 W, 100 W, 50 W, 25 W, 21 W, 15 W, 12 W, and/or any other appropriate power. Combinations of the foregoing ranges are contemplated including a peak power between 9 W and 130 W, between 9 W and 100 W, between 9 W and 50 W, between 9 W and 25 W, between 9 W and 21 W, between 9 W and 15 W, between 9 W and 12 W, between 10 W and 130 W, between 10 W and 100 W, between 10 W and 50 W, between 10 W and 25 W, between 10 W, and 21 W, between 10 W and 15 W, between 12 W and 130 W, between 12 W and 100 W, between 12 W and 50 W, between 12 W and 25 W, between 12 W and 21 W, between 12 W and 15 W between 15 W and 130 W, between 15 W and 100 W, between 15 W and 50 W, between 15 W and 25 W, between 15 W and 21 W, between 20 W and 130 W, between 20 W and 100 W, between 20 W and 50 W, between 20 W and 21 W, between 25 W and 130 W, between 25 W and 100 W, between 25 W and 50 W, between 50 W and 130 W, between 50 W and 100 W, or between 100 W and 130 W. As described herein, the phrase “between one value and another value” includes the endpoints and all values between the endpoints. In some instances, the above powers may be appropriate for forming a depot in the submucosal tissue and/or muscularis layer of the stomach of a subject. Alternatively, embodiments in which a drug delivery device is configured to provide a jet for intraluminal delivery where a majority of the active pharmaceutical ingredient is injected into the intraluminal space of the stomach are also contemplated. In such an embodiment, a peak power of the jet may be less than 9 W. Additionally, embodiments in which a drug delivery device is configured for intraperitoneal delivery where a majority of the active pharmaceutical ingredient is injected into the peritoneal space by perforating the muscularis layer of the stomach are so contemplated. In some embodiments, an intraperitoneal injection within the stomach may correspond to jets with peak powers greater than about 40 W.

As also noted above, in some embodiments, it is desirable to deliver an active pharmaceutical ingredient to a tissue of the small intestine of a subject. Accordingly, an appropriate peak power may be selected to allow a jet to penetrate tissue of the small intestine proximate to a drug delivery device disposed within a small intestine of a subject. In some instances, the peak power may be selected to avoid perforating a muscularis layer of the small intestine. In such an embodiment, a peak power of a jet oriented towards a surface of a subject's small intestine may be greater than or equal to 3.0 W, 3.1 W, 3.2 W, 3.3 W, 3.4 W, 3.5 W, 4.0 W, 4.5 W, 5 W, 5.5 W, 6.0 W and/or any other appropriate power. Correspondingly, a peak power of the jet may be less than or equal to 6.5 W, 6.4 W, 6.3 W, 6.2 W, 6.1 W, 6.0 W, 5.5 W, 5.0 W, 4.5 W, and/or any other appropriate power. Combinations of the foregoing ranges are contemplated including a peak power between 3.0 W and 6.5 W, between 3.0 W and 6.4 W, between 3.0 W and 6.3 W, between 3.0 W and 6.2 W, between 3.0 W and 6.1 W, between 3.0 W and 6.0 W, between 3.0 W and 5.5 W, between 3.0 W and 5.0 W, between 3.0 W and 4.5 W, between 3.1 W and 6.5 W, between 3.1 W and 6.4 W, between 3.1 W and 6.3 W, between 3.1 W and 6.2 W, between 3.1 W and 6.1 W, between 3.1 W and 6.0 W, between 3.1 W and 5.5 W, between 3.1 W and 5.0 W, between 3.1 W and 4.5 W, between 3.2 W and 6.5 W, between 3.2 W and 6.4 W, between 3.2 W and 6.3 W, between 3.2 W and 6.2 W, between 3.2 W and 6.1 W, between 3.2 W and 6.0 W, between 3.2 W and 5.5 W, between 3.2 W and 5.0 W, between 3.2 W and 4.5 W, between 3.3 W and 6.5 W, between 3.3 W and 6.4 W, between 3.3 W and 6.3 W, between 3.3 W and 6.2 W, between 3.3 W and 6.1 W, between 3.3 W and 6.0 W, between 3.3 W and 5.5 W, between 3.3 W and 5.0 W, between 3.3 W and 4.5 W, between 3.4 W and 6.5 W, between 3.4 W and 6.4 W, between 3.4 W and 6.3 W, between 3.4 W and 6.2 W, between 3.4 W and 6.1 W, between 3.4 W and 6.0 W, between 3.4 W and 5.5 W, between 3.4 W and 5.0 W, between 3.4 W and 4.5 W, between 3.5 W and 6.5 W, between 3.5 W and 6.4 W, between 3.5 W and 6.3 W, between 3.5 W and 6.2 W, between 3.5 W and 6.1 W, between 3.5 W and 6.0 W, between 3.5 W and 5.5 W, between 3.5 W and 5.0 W, between 3.5 W and 4.5 W, between 4.0 W and 6.5 W, between 4.0 W and 6.4 W, between 4.0 W and 6.3 W, between 4.0 W and 6.2 W, between 4.0 W and 6.1 W, between 4.0 W and 6.0 W, between 4.0 W and 5.5 W, between 4.0 W and 5.0 W, between 4.0 W and 4.5 W, between 4.5 W and 6.5 W, between 4.5 W and 6.4 W, between 4.5 W and 6.3 W, between 4.5 W and 6.2 W, between 4.5 W and 6.1 W, between 4.5 W and 6.0 W, between 4.5 W and 5.5 W, between 4.5 W and 5.0 W, between 5.0 W and 6.5 W, between 5.0 W and 6.4 W, between 5.0 W and 6.3 W, between 5.0 W and 6.2 W, between 5.0 W and 6.1 W, between 5.0 W and 6.0 W, between 5.0 W and 5.5 W, between 5.5 W and 6.5 W, between 5.5 W and 6.4 W, between 5.5 W and 6.3 W, between 5.5 W and 6.2 W, between 5.5 W and 6.1 W, between 5.5 W and 6.0 W, between 6.0 W and 6.5 W, between 6.0 W and 6.4 W, between 6.0 W and 6.3 W, between 6.0 W and 6.2 W, between 6.0 W and 6.1 W, and/or any other appropriate range of peak powers. In some cases, a jetting power between 3.5 W and 6.5 W or between 4.0 W and 6.5 W may be preferable, as those jetting powers may have higher injection efficiency than other jetting powers. In some instances, the above powers may be appropriate for forming a depot in the submucosal tissue and/or muscularis layer of the small intestine of a subject. Alternatively, embodiments in which a drug delivery device is configured to provide a jet for intraluminal delivery where a majority of the active pharmaceutical ingredient is injected into the intraluminal space of the small intestine are also contemplated. In such an embodiment, a peak power of the jet may be less than 3.0 W. Additionally, embodiments in which a drug delivery device is configured for intraperitoneal delivery where a majority of the active pharmaceutical ingredient is injected into the peritoneal space by perforating the muscularis layer of the small intestine are also contemplated. In some embodiments, an intraperitoneal injection within the small intestine may correspond to jets with peak powers greater than about 6.5 W, 7.0 W, and/or any other appropriate power range.

The efficiency of depot formation in a target tissue may depend on the particular target tissue and the operating parameters applied when directing a jet of an active pharmaceutical ingredient towards the tissue. As used herein, a dosage of an API refers to the amount of the API initially contained within a drug delivery device. Depot efficiency refers to the percentage of the amount of the API initially contained within a drug delivery device that is subsequently delivered into the depot disposed within the target tissue. For example, a depot may be formed in the submucosal tissue and/or muscularis tissue of the stomach and/or small intestine of a subject. As elaborated on below, by appropriately selecting the operating parameters of the jet, depot efficiencies greater than 40% may be achieved. For instance, in some embodiments, a depot efficiency of a drug delivery device may be greater than or equal to 40%, 50%, 60%, 70%, and/or any other appropriate percentage. Correspondingly, a depot efficiency of a drug delivery device may be less than or equal to 95%, 90%, 80%, 70%, 60%, and/or any other appropriate percentage. Combinations of the foregoing are contemplated including depot efficiencies between 40% and 95%, 50% and 95%, 60% and 95%, 70% and 95%, 40% and 90%, 50% and 90%, 60% and 90%, 70% and 90%, 40% and 80%, 50% and 80%, 60% and 80%, 70% and 80%, 40% and 70%, 50% and 70%, 60% and 70%, 40% and 60%, 50% and 60%, and/or in the other appropriate combination. Additionally, it should be understood that depot efficiencies both greater than and less than those noted above are possible as the disclosure is not limited in this fashion.

Depending on the particular API being administered to a subject, a drug delivery device of exemplary embodiments described herein may be configured to deliver a range of different dose volumes of the API to a subject. According to exemplary embodiments described herein, a drug delivery device may include an API reservoir volume with the API disposed therein that is less than or equal to 500 μL, 300 μL, 200 μL, 150 μL, 100 μL, 75 μL, 50 μL, 25 μL, 10 μL, and/or any other appropriate volume. Correspondingly, a drug delivery device may contain an API reservoir volume greater than or equal to 1 μL, 5 μL, 10 μL, 25 μL, 50 μL, 75 μL, 100 μL, 200 μL, 300 μL, and/or any other appropriate volume. Combinations of the above-noted volumes are contemplated, including, but not limited to, reservoir volumes between 1 μL and 500 μL, between 1 μL, and 300 μL, between 1 μL and 200 μL, between 1 μL and 150 μL, between 1 μL and 100 μL, between 1 μL and 75 μL, between 1 μL and 50 μL, between 1 μL and 25 μL, between 1 μL and 10 μL, between 10 μL, and 500 μL, between 10 μL, and 300 μL, between 10 μL, and 200 μL, between 10 μL, and 150 μL, between 10 μL, and 100 μL, between 10 μL, and 75 μL, 10 μL, and 50 μL, between 10 μL, and 25 μL, between 25 μL, and 500 μL, between 25 μL, and 300 μL, between 25 μL, and 200 μL, between 25 μL, and 150 μL, between 25 μL, and 100 μL, between 25 μL, and 75 μL, between 25 μL, and 50 μL, between 50 μL, and 500 μL, between 50 μL, and 300 μL, between 50 μL, and 200 μL, between 50 μL, and 150 μL, between 50 μL, and 100 μL, between 50 μL, and 75 μL, between 75 μL, and 500 μL, between 75 μL, and 300 μL, between 75 μL, and 200 μL, between 75 μL, and 150 μL, between 75 μL, and 100 μL, between 100 μL, and 500 μL, between 100 μL, and 300 μL, between 100 μL, and 200 μL, between 100 μL, and 150 μL, between 150 μL, and 500 μL, between 150 μL, and 300 μL, between 150 μL, and 200 μL, between 200 μL, and 500 μL, between 200 μL, and 300 μL, or between 300 μL, and 500 μL. Of course, any suitable reservoir volume may be employed in a drug delivery device as the present disclosure is not so limited. Additionally, depots of an API disposed in a target tissue may have a volume that is related to the above noted volumes by the corresponding depot efficiencies described above.

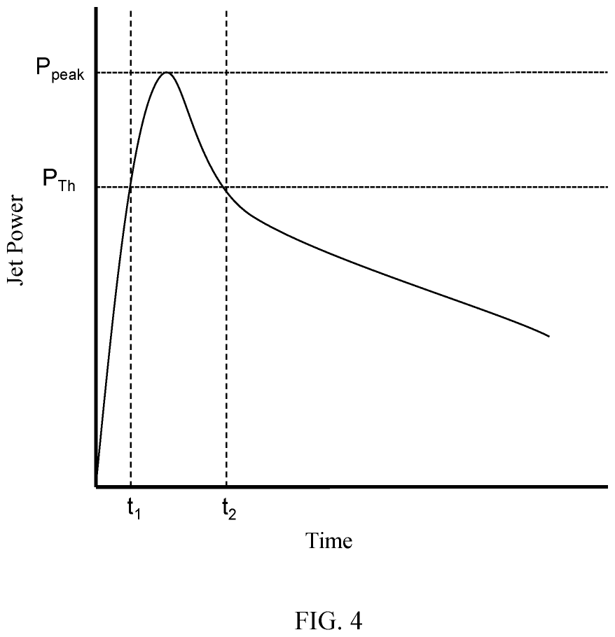

In order to form an efficient depot within a target tissue, it may be desirable to maintain a power of a jet within a predetermined range of a peak power of the jet for a predetermined time period. As defined herein, and as shown in FIG. 4, a peak power (Pₚₑₐₖ) of a jet refers to the maximum power of the jet. A threshold power refers to the minimum power of a jet required to penetrate a target tissue at a location within a gastrointestinal tract of a subject. In some embodiments, the peak power is greater than or equal to the threshold power. An “optimal peak power” refers to the minimum peak power of a jet appropriate for forming a desired depot with a depot efficiency of at least 50% in a target tissue at a location within the gastrointestinal tract of a subject. For example, a power of the jet may be maintained within 5%, 10%, or other appropriate percentage of the peak power for the predetermined time period. For example, as shown in FIG. 4, the jet power may initially increase until it is greater than a threshold power P_{Th} at time t₁ after which the power may continue to increase to the peak power Pₚₑₐₖ. The jet power may then decrease until it is equal to the threshold power at time t₂ where the predetermined time period corresponds to the difference between times t₁ and t₂. The power may continue to decrease after this time as shown in the figure. Depending on the particular application, the predetermined time period may be greater than or equal to 1 ms, 10 ms, 50 ms, 100 ms, and/or any other appropriate time period. Correspondingly, the predetermined time period may be less than or equal to 300 ms, 200 ms, 100 ms, 50 ms, and/or any other appropriate time period. Combinations of the foregoing are contemplated including, for example, a predetermined time period that is between 1 ms and 300 ms, between 1 ms and 200 ms, between 1 ms and 100 ms, between 1 ms and 50 ms, between 10 ms and 300 ms, between 10 ms and 200 ms, between 10 ms and 100 ms, between 10 ms and 50 ms, between 50 ms and 300 ms, between 50 ms and 200 ms, between 50 ms and 100 ms, between 100 ms and 300 ms, or between 100 ms and 200 ms. Of course, it should be understood that predetermined time periods and appropriate ranges of the jet power relative to the peak power other than those noted above are also contemplated as the disclosure is not so limited.

According to exemplary embodiments described herein, a trigger of a drug delivery device may be configured to actuate the drug delivery device in the GI tract of a subject at a predetermined time and/or location in the GI tract. In some embodiments, the trigger may be a passive component configured to interact with the environment of the GI tract to actuate the drug delivery device. For example, in some embodiments the trigger may be a sugar plug, or other dissolvable material, configured to dissolve in the GI tract. The dissolvable plug may have a certain thickness and/or shape that at least partly determines the speed at which the dissolvable plug dissolves and ultimately actuates the drug delivery device. In another embodiment, the trigger may be at least partially formed by an enteric coating. For example, in some embodiments, a trigger may include both a dissolvable plug and an enteric coating disposed on an exterior surface of the dissolvable plug, as the present disclosure is not so limited. Other appropriate materials for a dissolvable trigger may include, but are not limited to, sugar alcohols such as disaccharides (e.g. Isomalt), water soluble polymers such as Poly-vinyl alcohol, enteric coatings, time-dependent coatings, enteric and time-dependent coatings, temperature-dependent coatings, light-dependent coatings, and/or any other appropriate material capable of being dissolved within the GI tract of a subject. In some embodiments, a trigger may include a triggerable membrane including ethylenediaminetetraacetic acid, glutathione, or another suitable chemical. In some embodiments, a sugar alcohol trigger may be employed in combination with an enteric coating configured to protect the sugar alcohol trigger until the drug delivery device is received in the GI tract of a subject. In other embodiments, the trigger may include a pH responsive coating to assist with delaying triggering until after ingestion. In some embodiments, the trigger may be a sensor and/or electrodes that are configured to either detect or interact with one or more characteristics of the GI tract to actuate the device. For example, a sensor detecting contact with a GI mucosal lining may be used to actuate the device. In embodiments where a sensor is employed, the trigger may also include an active component that moves, or is otherwise actuated, in response to a predetermined condition being detected by the sensor. For example, a gate may be moved when contact with a GI mucosal tract is detected. In other embodiments the trigger may employ electrical power to melt or weaken a rupturable membrane (e.g., by applying a voltage across a conductive rupturable membrane) and/or trigger a chemical reaction. Of course, any suitable active or passive trigger may be employed for a drug delivery device, as the present disclosure is not so limited.

According to exemplary embodiments described herein, a drug delivery device includes a potential energy source which is used to store energy in the drug delivery device that is used to generate a jet of an API when the drug delivery device is actuated. In some embodiments, the potential energy source may be a compressed gas. The compressed gas may be directly stored in the drug delivery device, or the compressed gas may be generated via a chemical reaction or phase change. For example, in some embodiments dry ice may be stored in a chamber of the drug delivery device so that compressed gas is generated as the dry ice sublimates. Alternatively, a compressed gas may be provided to a desired chamber prior to sealing a drug delivery device. In some embodiments, the potential energy source may be a spring (e.g., a compressed compression spring). In some embodiments, the potential energy source may be a reaction chamber. For example, the reaction chamber may allow an acid and base to be combined to generate gas, leading to the expulsion of API from the drug delivery device when the device is actuated. Alternatively, in another embodiment, a trigger may detonate an explosive material located within a chamber to generate pressurized gas for expelling the API from the drug delivery device. Of course, any suitable reaction or other potential energy source may be employed to pressurize and drive an API in a jet when a drug delivery device is actuated, as the present disclosure is not so limited.

As noted above, jetting power may be tuned to deliver an API into different target tissues within the GI tract with different penetration characteristics. Jetting power may be at least partly determined by jet velocity, fluid density and jet diameter. Accordingly, a drug delivery device according to exemplary embodiments described herein may be appropriately sized and include an appropriate amount of potential energy to generate a jet with enough power to deliver an API into the tissue of the GI tract in a desired location.

To achieve the exemplary jetting powers described herein, a jet generated by a drug delivery device of exemplary embodiments described herein may have a corresponding velocity. Accordingly, in some embodiments, a drug delivery device may be configured to generate a jet having a velocity less than or equal to 250 m/s, 200 m/s, 150 m/s, 130 m/s, 100 m/s, 75 m/s, 50 m/s and/or another appropriate velocity. Correspondingly, a drug delivery device may be configured to generate a jet having a velocity greater than or equal to 20 m/s, 30 m/s, 50 m/s, 80 m/s, 100 m/s, 150 m/s, 200 m/s, and/or another appropriate velocity. Combinations of the above-noted ranges are contemplated, including, but not limited to, jet velocities between 20 m/s and 250 m/s, between 20 m/s and 200 m/s, between 20 m/s and 100 m/s, between 20 m/s and 150 m/s, between 20 m/s and 100 m/s, between 20 m/s and 75 m/s, between 20 m/s and 50 m/s, between 50 m/s and 250 m/s, between 50 m/s and 200 m/s, between 50 m/s and 100 m/s, between 50 m/s and 150 m/s, between 50 m/s and 100 m/s, between 50 m/s and 75 m/s, between 75 m/s and 250 m/s, between 75 m/s and 200 m/s, between 75 m/s and 100 m/s, between 75 m/s and 150 m/s, between 75 m/s and 100 m/s, between 100 m/s and 250 m/s, between 100 m/s and 200 m/s, between 100 m/s and 150 m/s, between 150 m/s and 250 m/s, between 150 m/s and 200 m/s, or between 200 m/s and 250 m/s. In one specific embodiment, the target tissue location may correspond to the stomach, and a jet velocity of the jet may preferably be between 80 m/s and 130 m/s, or between 40 m/s and 60 m/s. In another embodiment, the target tissue location may correspond to the small intestine of a subject, and a jet velocity of the jet may preferably be between 40 m/s and 80 m/s. Of course, any jet velocity suitable to deliver an API into a corresponding tissue of the gastrointestinal tract of a subject may be used as the present disclosure is not so limited.

In some embodiments, a maximum transverse dimension (e.g. diameter) of an outlet, such as a nozzle a jet is emitted from, and/or a maximum transverse dimension (e.g. diameter) of a jet emitted from the outlet may be less than or equal to 550 μm, 450 μm, 400 μm, 350 μm, 300 μm, 250 μm, 200 μm, 150 μm, 100 μm, 75 μm, 50 μm, 25 μm, 10 μm, and/or any other appropriate dimension. Correspondingly, a maximum transverse dimension of the outlet and/or jet may be greater than or equal to 5 μm, 10 μm, 25 μm, 50 μm, 75 μm, 100 μm, 150 μm, 200 μm, 250 μm, 300 μm, and/or any other appropriate dimension. Combinations of the above-noted ranges are contemplated, including, but not limited to, maximum transverse dimensions of a jet and/or outlet between 5 μm and 550 μm, between 5 μm and 450 μm, between 10 μm and 450 μm, between 25 μm and 450 μm, between 50 μm and 450 μm, between 75 μm and 450 μm, between 100 μm and 450 μm, between 150 μm and 450 μm, between 200 μm and 450 μm, between 250 μm and 450 μm, between 300 μm and 450 μm, between 5 μm and 400 μm, between 10 μm and 400 μm, between 25 μm and 400 μm, between 50 μm and 400 μm, between 75 μm and 400 μm, between 100 μm and 400 μm, between 150 μm and 400 μm, between 200 μm and 400 μm, between 250 μm and 400 μm, between 300 μm and 400 μm, between 5 μm and 350 μm, between 10 μm and 350 μm, between 25 μm and 350 μm, between 50 μm and 350 μm, between 75 μm and 350 μm, between 100 μm and 350 μm, between 150 μm and 350 μm, between 200 μm and 350 μm, between 250 μm and 350 μm, between 300 μm and 350 μm, between 5 μm and 300 μm, between 10 μm and 300 μm, between 25 μm and 300 μm, between 50 μm and 300 μm, between 75 μm and 300 μm, between 100 μm and 300 μm, between 150 μm and 300 μm, between 200 μm and 300 μm, between 250 μm and 300 μm, between 5 μm and 250 μm, between 10 μm and 250 μm, between 25 μm and 250 μm, between 50 μm and 250 μm, between 75 μm and 250 μm, between 100 μm and 250 μm, between 150 μm and 250 μm, between 200 μm and 250 μm, between 5 μm and 200 μm, between 10 μm and 200 μm, between 25 μm and 200 μm, between 50 μm and 200 μm, between 75 μm and 200 μm, between 100 μm and 200 μm, between 150 μm and 200 μm, between 5 μm and 150 μm, between 10 μm and 150 μm, between 25 μm and 150 μm, between 50 μm and 150 μm, between 75 μm and 150 μm, between 100 μm and 150 μm, between 5 μm and 100 μm, between 10 μm and 100 μm, between 25 μm and 100 μm, between 50 μm and 100 μm, between 75 μm and 100 μm, between 5 μm and 75 μm, between 10 μm and 75 μm, between 25 μm and 75 μm, between 50 μm and 75 μm, between 5 μm and 50 μm, between 10 μm and 50 μm, between 25 μm and 50 μm, between 5 μm and 25 μm, between 10 μm and 25 μm, or between 5 μm and 10 μm. Of course, any suitable dimension of an outlet and/or jet suitable for delivery of an API into the tissue of a desired portion of a gastrointestinal tract of a subject may be employed as the present disclosure is not so limited.

According to exemplary embodiments described herein, a drug delivery device includes a potential energy source configured to pressurize an API so that the API may be released in a jet into a GI tract mucosal lining. The pressure applied to the reservoir may affect jetting power and/or a jet velocity of an API jet emitted by the drug delivery device. In some embodiments, the potential energy source may be configured to apply a pressure to an API reservoir less than or equal to 1000 bar, 800 bar, 600 bar, 500 bar, 250 bar, 100 bar, 60 bar, 45 bar, 40 bar, 10 bar, 1 bar, and/or any other appropriate pressure. Correspondingly, the potential energy source may apply a pressure to an API reservoir greater than or equal to 0.1 bar, 1 bar, 10 bar, 15 bar, 20 bar, 40 bar, 45 bar, 60 bar, 100 bar, 250 bar, 500 bar, 600 bar, 800 bar, and/or any other appropriate pressure. Combinations of the above-noted ranged are contemplated, including, but not limited to, pressures between 0.1 bar and 1000 bar, between 0.1 bar and 800 bar, between 0.1 bar and 600 bar, between 0.1 bar and 500 bar, between 0.1 bar and 250 bar, between 0.1 bar and 100 bar, between 0.1 bar and 60 bar, between 0.1 bar and 40 bar, between 0.1 bar and 10 bar, between 0.1 bar and 1 bar, between 1 bar and 1000 bar, between 1 bar and 800 bar, between 1 bar and 600 bar, between 1 bar and 500 bar, between 1 bar and 250 bar, between 1 bar and 100 bar, between 1 bar and 60 bar, between 1 bar and 40 bar, between 1 bar and 10 bar, between 10 bar and 1000 bar, between 10 bar and 800 bar, between 10 bar and 600 bar, between 10 bar and 500 bar, between 10 bar and 250 bar, between 10 bar and 100 bar, between 10 bar and 60 bar, between 10 bar and 40 bar, between 10 bar and 800 bar, between 10 bar and 600 bar, between 10 bar and 500 bar, between 10 bar and 250 bar, between 10 bar and 100 bar, between 10 bar and 60 bar, between 10 bar and 40 bar, between 40 bar and 800 bar, between 40 bar and 600 bar, between 40 bar and 500 bar, between 40 bar and 250 bar, between 40 bar and 100 bar, between 40 bar and 60 bar, between 60 bar and 800 bar, between 60 bar and 600 bar, between 60 bar and 500 bar, between 60 bar and 250 bar, between 60 bar and 100 bar, between 100 bar and 800 bar, between 100 bar and 600 bar, between 100 bar and 500 bar, between 100 bar and 250 bar, between 250 bar and 800 bar, between 250 bar and 600 bar, between 250 bar and 500 bar, between 500 bar and 800 bar, between 500 bar and 600 bar, or between 600 bar and 800 bar. In some embodiments, a pressure applied to the API reservoir of between 15 bar and 60 bar, and more preferably between 15 bar 45 bar, may be especially efficacious at forming high-efficiency depots in the submucosal tissue of the stomach when combined with appropriately sized nozzles. Similarly, a pressure applied to the API reservoir of between 10 bar and 20 bar may be effective in forming high-efficiency depots in the submucosal tissue of the intestines of a subject when combined with appropriately sized nozzles. Of course, any suitable pressure may be applied to an API reservoir as the present disclosure is not so limited.

In some embodiments, a drug delivery device is sized and shaped to be ingested by a subject. Accordingly, the drug delivery device may be appropriately small so that the drug delivery device may be easily swallowed and subsequently pass through the GI tract including the esophagus and pyloric orifice within the stomach. In some embodiments, a drug delivery device may include an overall length, such as a maximum dimension along a longitudinal axis of the device, that is less than or equal to 40 mm, 30 mm, 20 mm, 10 mm, 5 mm, and/or another appropriate length. Correspondingly, a drug delivery device may have an overall length greater than or equal to 3 mm, 5 mm, 10 mm, 20 mm, 25 mm, and/or another appropriate length. Combinations of the above-noted ranges are contemplated, including, but not limited to, overall lengths between 5 mm and 30 mm, 10 mm and 30 mm, 5 mm and 20 mm, as well as 5 mm and 10 mm. In some embodiments, a drug delivery device may have a maximum external transverse dimension, such as a diameter or other dimension that may be perpendicular to the longitudinal axis, that is less than or equal to 11 mm, 10 mm, 7 mm, 5 mm, and/or another appropriate dimension. Correspondingly, a drug delivery device may have a maximum external transverse dimension greater than or equal to 3 mm, 5 mm, 7 mm, 9 mm, and/or another appropriate dimension. Combinations of the above-noted ranges are contemplated, including, but not limited to, maximum external transverse dimensions between 3 mm and 11 mm, between 3 mm and 10 mm, between 3 mm and 7 mm, between 3 mm and 5 mm, and between 5 mm and 11 mm. In some embodiments, a drug delivery device may have an overall volume less than or equal to 3500 mm³, 3000 mm³, 2500 mm³, 2000 mm³, 1500 mm³, 1000 mm³, 750 mm³, 500 mm³, 250 mm³, 100 mm³, and/or any other appropriate volume. Corresponding, a drug delivery device may have an overall volume greater than or equal to 50 mm³, 100 mm³, 250 mm³, 500 mm³, 750 mm³, 1000 mm³, 1500 mm³, 2000 mm³, 2500 mm³, and/or any other appropriate volume. Combinations of the above-noted ranged are contemplated, including, but not limited to, volumes between 1000 mm³ and 3000 mm³, 1500 mm³ and 3000 mm³, 50 mm³ and 500 mm³, 50 mm³ and 100 mm³, as well as 2000 mm³ and 3000 mm³. Of course, any suitable overall length, maximum external transverse dimension, and volume for an ingestible delivery device may be employed, as the present disclosure is not so limited.

According to exemplary embodiments described herein, the drug delivery device is administered to a subject orally. In other embodiments, the drug delivery device may be administered, rectally, endoscopically, or nasally, as the present disclosure is not so limited. Accordingly, it should be understood that the currently disclosed drug delivery devices may be delivered to a desired portion of the gastrointestinal tract of a subject in a number of different ways, and the current disclosure is not limited to the specific method of deploying the drug delivery device.

In some embodiments, it may be desirable to position an outlet of a jet proximate to a surface of the GI tract of a subject and/or to orient the outlet towards the surface prior to actuating a delivery device to help ensure delivery of an API into the desired tissue. Accordingly, depending on the particular embodiment, a variety of different strategies may be employed. For example, various mucoadhesives, dissolvable hooks for attaching to tissue, mucosal contact sensors, self-orienting delivery devices (e.g. buoyancy and/or center of gravity based orientation systems), and other methods of either maintaining a delivery device in contact with and/or determining when they delivery device is proximate to and/or oriented towards a desired tissue within a GI tract may be used. For example, various self-righting or self-orienting structures and/or methods described in WO 2018/213600 A1 can be employed by the drug delivery device in accordance with the present disclosure. WO 2018/213600 A1 is incorporated herein by reference in its entirety. Additionally, in some embodiments, multiple outlets and corresponding multiple jets located at different positions on an exterior of the delivery device may be used to increase the chance of one of the jets being oriented towards a tissue proximate to the delivery device. Of course, it should be understood that embodiments in which a delivery device does not include sensors for sensing contact with and/or a component for attaching to a mucosal lining of a subject are also contemplated.

In embodiments in which systems and/or methods are used to actuate delivery of an API when an outlet is oriented towards gastrointestinal tissue proximate to the drug delivery device, it may be desirable to maintain an orientation of the outlet and corresponding jet within a predetermined range of angles relative to the underlying tissue. This may help to provide a desired combination of force and/or power of the jet in a direction oriented perpendicular to a surface of the adjacent tissue. For example, an angle of a jet emitted from an outlet relative to a direction normal to the underlying tissue surface may be less than or equal to 20°, 15°, 10°, 5°, and/or any other appropriate range of angles including angles both greater than and less than those noted above. The above-noted angular relationship of the jet direction emitted from an outlet of a device versus a direction normal to the underlying tissue surface may be provided using any of the above noted methods and structures for actuating a delivery device while in a desired orientation relative to the underlying tissue.

In some embodiments, a jet may be emitted from an outlet that is distanced from a tissue underlying a delivery device that the jet impinges on. The Inventors have recognized that for standoff distances between an outlet and an underlying tissue less than a threshold distance, minimal differences in tissue penetration and API delivery have been noted. As defined herein, a standoff distance refers to the shortest distance between an outlet and the surface of an underlying tissue that a jet projected from the outlet impinges upon. Accordingly, in some embodiments, a standoff distance may be less than or equal to 10 mm, 7.5 mm, 5 mm, 4 mm, 3 mm, 2 mm, 1 mm, and/or any other appropriate distance. While particular ranges of distances are given above, it should be understood that acceptable standoff distances between the outlet and the underlying tissue may vary depending on the specific jet parameters and tissue being deployed into as well as the specific application the drug delivery device is being used for. Accordingly, standoff distances both greater than and less than those noted above are contemplated as the disclosure is not so limited.

In some embodiments, it may be desirable to form one or more components of a drug delivery device from a biocompatible and/or bio inert material. For example, various components may be exposed to the fluids and/or solids present within the gastrointestinal tract of a subject when ingested. Accordingly, components that may be exposed to the fluids and/or solids present within the gastrointestinal tract may be made from materials including, but not limited to: metals that are relatively inert to the gastrointestinal environment such as titanium; non-toxic and/or inert polymers such as polydimethylsiloxane (PDMS), polycaprolactone (PCL); salts; carbohydrates; and/or any other appropriate material for a desired application. In instances where a particular component used in a delivery device may be inappropriate for exposure to the environment within the gastrointestinal system of a subject, a non-reactive polymeric and/or metallic coating may be applied to the component to isolate the underlying material from the exterior environment. Alternatively, such a component may be contained within a portion of the delivery device that is not exposed to the exterior environment during operation. In view of the above, it should be understood that the various components disclosed herein may be made using any appropriate combination materials as the disclosure is not limited to any particular construction and/or combination of materials of the various components.

As used herein, the term “active pharmaceutical ingredient” (also referred to as a “drug” or “therapeutic agent”) refers to an agent that is administered to a subject to treat a disease, disorder, or other clinically recognized condition, or for prophylactic purposes, and has a clinically significant effect on the body of the subject to treat, prevent, and/or diagnose the disease, disorder, or condition. The active pharmaceutical ingredient may be delivered to a subject in a quantity greater than a trace amount to affect a therapeutic response in the subject. In some embodiments, active pharmaceutical ingredients (APIs) can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals. Certain such APIs may include molecules such as proteins, peptides, hormones, nucleic acids, gene constructs, etc., for use in therapeutic, diagnostic, and/or enhancement areas. In certain embodiments, the API is a small molecule and/or a large molecule. Accordingly, it should be understood that the API's described herein are not limited to any particular type of API. Additionally, while according to exemplary embodiments described herein a drug delivery device may deliver an API in the form of an incompressible liquid jet, in other embodiments a jet including an API generated by a drug delivery device may be formed of gases, viscous fluids, aerosolized powders, and/or other appropriate materials, as the present disclosure is not so limited.

In some embodiments, as used herein, a jet may refer to a collimated flow of gas, fluid, aerosolized powder, combinations of the forgoing, and/or other appropriate materials.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

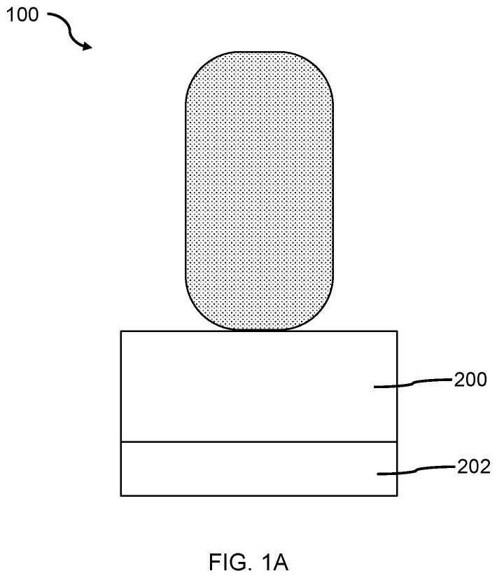

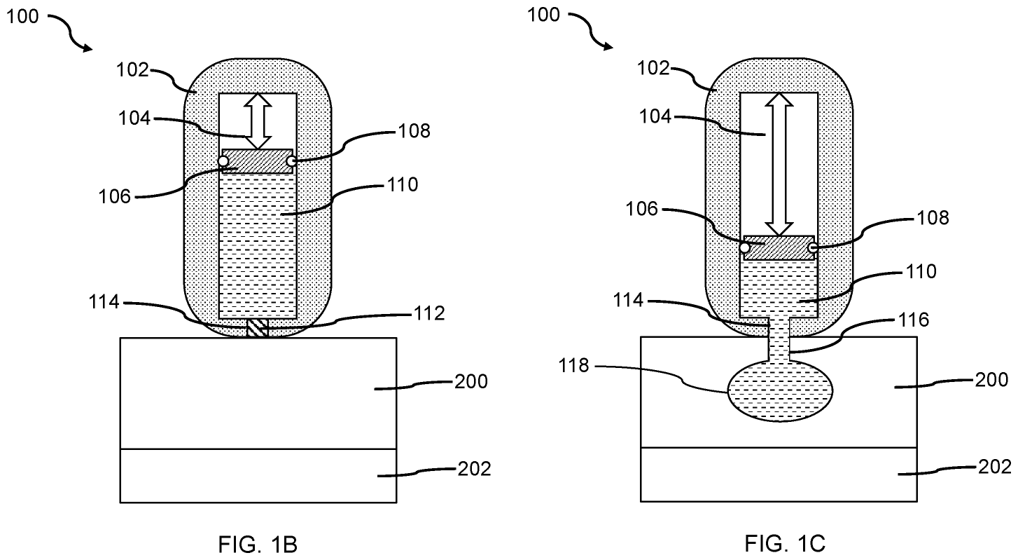

FIGS. 1A-1C depict a schematic of one embodiment of a drug delivery device 100. As shown in FIG. 1B, the drug delivery device 100 includes a housing 102 containing a potential energy source configured as a compressed gas compartment 104 and an active pharmaceutical ingredient (API) reservoir 110. The compressed gas compartment 104 and reservoir 110 are separated by a piston 106 slidably received in an interior of the housing 102 between the gas compartment 104 and the reservoir 110. The piston 106 includes a piston seal 108 configured to inhibit fluid transfer between the compressed gas compartment 104 and the reservoir 110. The piston 106 transfers pressure from the compressed gas compartment 104 to the reservoir 110. That is, the compressed gas inside of the gas compartment 104 pressurizes the API disposed inside of the reservoir 110. As shown in FIG. 1A, the reservoir 110 includes an outlet 114 in fluid communication with an exterior environment of the device. In some embodiments, the outlet 114 may function as a nozzle for formation of the jet 116. The outlet's maximum transverse dimension (e.g. a diameter) may be selected to provide a desired maximum transverse dimension of a corresponding jet 116 that is emitted from the outlet 114 when the pressurized API flows out of the reservoir 110.

The device may also include a trigger 112 that is operatively associated with the potential energy source, which in this case is the pressurized gas compartment 104. The trigger 112 is configured to actuate the device 100 at a predetermined location within the gastrointestinal tract of a subject such that the potential energy source, which in the current embodiment is the compressed gas compartment 104, compresses the reservoir 110 to deploy a jet 116 of the active pharmaceutical ingredient out of the outlet 114 and into tissue 200 of a corresponding portion of the gastrointestinal tract located proximate to the device and that the outlet 114 is oriented towards. For example, in the depicted embodiment, the trigger 112 may correspond to a dissolvable plug physically retained within the outlet 114 of the device such that when the trigger 112 is dissolved the device actuates as elaborated on below, though any appropriate trigger may be used as the disclosure is not so limited.

As shown in FIG. 1C, when the trigger 112 is dissolved, or otherwise actuated inside of a GI tract of the subject, the barrier preventing deployment of the API through the outlet 114 is removed. Accordingly, the pressure applied to the API reservoir 110 by the piston 106 associated with the compressed gas compartment 104 causes the piston 106 to move in a direction that compresses the reservoir 110. As the reservoir 110 is compressed, the API flows out of the outlet 114 in the form of a jet 116 with sufficient velocity to penetrate tissue 200 of the gastrointestinal tract that is located proximate to the outlet 114. Again, the depicted tissue 200 of the gastrointestinal tract may correspond to the stomach, small intestine, and/or any other anatomical structure of the gastrointestinal tract of a subject described herein. Depending on the embodiment, the jet 116 may form a depot 118 of the API within the tissue 200 of the gastrointestinal tract without perforating the gastrointestinal tract. For example, the outlet 114, API reservoir 110, and associated potential energy source (e.g. the compressed gas compartment 104) may be appropriately configured to provide a jet that is optimized to form a depot corresponding to a volume of the API disposed within the submucosal tissue of the gastrointestinal tract without perforating a muscularis layer 202 of the gastrointestinal tract. Further, depending on the specific operating parameters, the depot 118 may be at least partially disposed in a submucosal tissue and/or the muscularis layer 202 of the gastrointestinal tract.

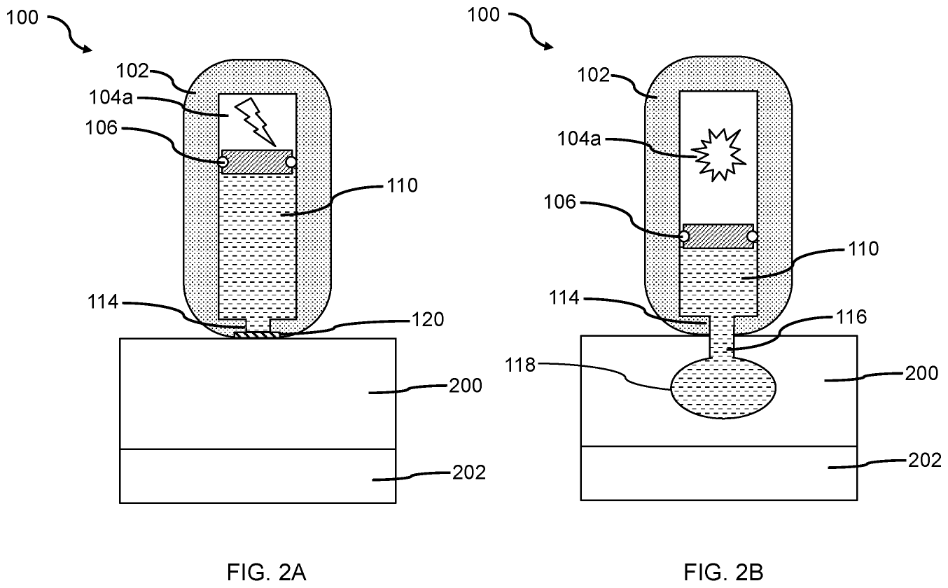

FIGS. 2A-2B depict a schematic embodiment of a drug delivery devices 100 including a different type of potential energy source and trigger. In the depicted embodiment, the trigger is based on a reaction instead of dissolution of a dissolvable plug. For example, the drug delivery device 100 may include a housing 102 having a reaction chamber 104a and an API reservoir 110. Like the embodiment of FIGS. 1A-1C, the device also includes a piston 106 configured to transfer pressure between the reaction chamber 104a and the API reservoir 110 such that the piston 106 compresses the reservoir 110 upon actuation. The API reservoir 110 may also be in fluid communication with an outlet 114. In some embodiments, a rupturable membrane 120, or other seal, is disposed on, in, or is otherwise associated with the outlet 114 to seal the API inside of the API reservoir 110 until the device is actuated and the membrane 120 is ruptured. In the depicted embodiment, the reaction chamber 104a is not pressurized in the state shown in FIG. 2A, such that pressure is not applied to the rupturable membrane 120 in a resting state. Instead, the trigger may be an electrical trigger (e.g., a sensor) and/or a chemical trigger that is actuated at a predetermined time and/or location within the gastrointestinal tract (e.g. within the stomach and/or small intestine) of a subject using any of the previously described methods. The reaction chamber 104a may include reactants configured to generate pressure when actuated by the trigger. In some embodiments, an electrical sensor may trigger an acid-base reaction, an explosive reaction, and/or any other appropriate reaction to generate pressurized gas. Of course, any suitable reactants may be used to generate pressure, as the present disclosure is not so limited. Of course, while a dissolving trigger is not used in the embodiment of FIGS. 2A-2B, in other embodiments a dissolving trigger may be employed with a reaction chamber 104a where the dissolvable trigger exposes the reaction chamber 104a to an external gastric environment upon dissolution such that a reactant may react to produce gas when exposed to the gastric environment.

As shown in FIG. 2B, when the reaction is triggered inside of the reaction chamber 104a to pressurize the reaction chamber 104a, the piston 106 is forced down to pressurize the API in the API reservoir 110 which ruptures the rupturable membrane 120 or other seal. The API is then forced out of the outlet 114 of the reservoir 110 in a jet 116 with enough power to penetrate the GI tract tissue 200 to deliver a therapeutic dose of the API to the patient as previously described.

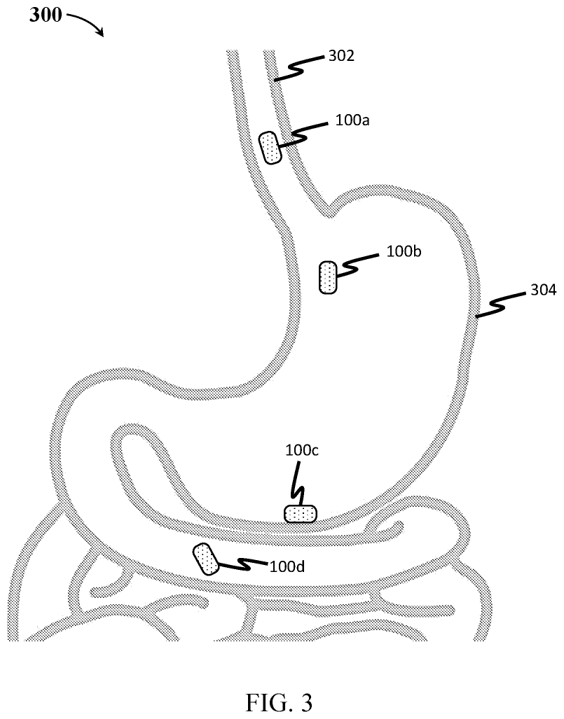

FIG. 3 depicts one embodiment of a drug delivery device 100 being orally ingested and passing through the gastrointestinal tract 300 of a subject. By way of example, and without wishing to be limited by such an exemplary set of embodiments, the system may be administered to a subject orally where it travels through the gastrointestinal tract 300 of the subject until it is actuated at a predetermined time and/or location within the gastrointestinal tract 300. For example, as illustrated schematically in FIG. 3, a drug delivery device 100 may be administered to a subject (e.g., orally) such that the device enters gastrointestinal tract 300 of the subject via the esophagus 302 (device 100a). The device may travel through gastrointestinal system until reaching the stomach 304 of the subject (device 100b). In some embodiments, the drug delivery device 100 may be denser than the surrounding fluids within the stomach 304, or other portion of the GI tract, causing the device to sink to the bottom of stomach 304 (device 100c) such that an external surface of the device contacts an internal surface of stomach 304. Depending on the embodiment, the device may either attach to the surface of the stomach 304 using an appropriate attachment method as previously described and/or the system may simply actuate without attaching to the tissue of the stomach 304. In either case, the device may self-actuate when at the appropriate location within the gastrointestinal tract 300 to deploy a jet of an active pharmaceutical ingredient into tissue of the gastrointestinal tract 300 located proximate to the device (e.g., the surface of the stomach). Subsequently, the device may pass through the pyloric orifice of the stomach 304 and through the remainder of the gastrointestinal tract 300 of the subject (device 100d). While FIG. 3 illustrates the operation of a device that deploys an active pharmaceutical ingredient within the stomach 304 of a subject, those of ordinary skill in the art would understand, based upon the teachings of this specification, that the drug delivery devices disclosed herein may deploy an active pharmaceutical ingredient at any desired location along the length of a gastrointestinal tract 300 of a subject, including the small intestine of the subject, and the jet may form a depot of the active pharmaceutical ingredient in any appropriate tissue of the target portion of the gastrointestinal tract 300 including, but not limited to, the mucosal, sub-mucosal, and/or muscularis tissue layer(s). As noted previously, in some embodiments, the jet may form the depot in the one or more layers of the tissue of the gastrointestinal tract without perforating the muscularis tissue of the gastrointestinal tract.

### Example: Gastrointestinal Tract Tissue Comparison

Table I below presents a comparison of characteristics of different gastrointestinal tissue anatomy. Generally GI tissue is composed of four broad cell layers: the mucosa, which secretes mucus, and acts as the first barrier to absorption of substances such as large molecules; the submucosa is which is disposed beneath the mucosa and is rich with vasculature for carrying nutrients to and from the mucosa; the muscularis which is disposed beneath the submucosa and is responsible for motility; and the serosa which is disposed beneath the muscularis and functions as the outermost, protective layer for each organ.

TABLE I

| | | | | | Overall | Bolus | |
|---|---|---|---|---|---|---|---|
| | | | | | Wall | Transit | |
| Organ | Length | | Diameter | | Thickness | Time | |
| Cheek (Buccal) | N/A | | N/A | | ~8 mm | ~30 | s |
| Esophagus | 18-25 | cm | ~2 | cm | 2-5 mm | 3 s-10 min | |
| Stomach | 26-3 | cm | 8-10 cm (max) | | 3-5 mm | 3-5 | hours |
| Small intestine | 6-8 | m | 2-4 | cm | 1-2 mm | 2-6 | hours |
| Large intestine | ~1.5 | m | 6-8 | cm | 1-5 mm | 10-60 | hours |

| | | | | | | |
|---|---|---|---|---|---|---|
| Rectum | 10-15 | cm | 4 cm (max) | 2-4 mm | 10-72 | hours |

In view of the above comparison of organ parameters, the stomach is an appealing target location due to the relatively long bolus transit time and larger wall thicknesses. Additionally, while the small intestinal wall may be relatively thinner (1-2 mm), the relatively small diameter of the small intestine makes it appealing for jet deployment of an API since all sides of a device would be in relatively close proximity to the intestinal wall. Accordingly, both the stomach and small intestine of a subject are appealing targets for deployment of an API using the jetting methods disclosed herein.

### Example: Jet Power

Due to the differences in the different tissue located along the length of the gastrointestinal tract of the subject, it is expected that each type of gastrointestinal tissue tissues will have different power requirements for the formation of a depot in a target tissue. Given these differences, it is not expected that a jet optimized for forming a depot in the stomach would be appropriate for forming a depot in the small intestine or other anatomical structure. When dealing with API's these differences, if not appropriately accounted for, could result in a dosage either not being delivered to the target tissue and/or unintentionally perforating one or more anatomical structures. Accordingly, it is desirable to both characterize how jets are deployed as well as the specific power requirements for forming a depot in a desired portion of the gastrointestinal tract of a subject to provide the desired amount of an API to the desired target tissue.

A model for the power of a jet emitted from a drug delivery device was developed. The model assumed the use of a linear compression spring as the potential energy source which was used to drive a piston for forcing fluid through a corresponding outlet. The spring was modeled as a linear spring with a stored compression force prior to deployment and a “dead” compression force after expansion and jet expulsion. The model did not account for friction. However, as discussed below, some energy may be lost to the friction imparted by the piston's sliding, and the flow constriction from the nozzle during actual usage. Bernoulli's equation was used for modeling the flow of the liquid jet expelled from the device where the fluid density was assumed to be 1000 kg/m3. The initial boundary conditions used to solve the model was the initial piston position at time zero and that the time required for acceleration of the piston was negligible (i.e. the velocity boundary condition was “free” at t=0). In order to improve the accuracy of the model, two types of friction losses were used including friction from the piston and nozzle efficiency loss.

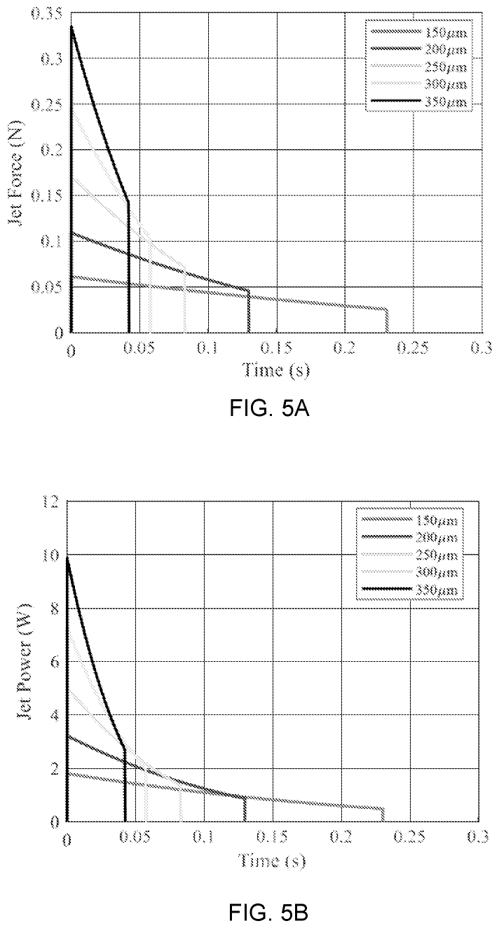

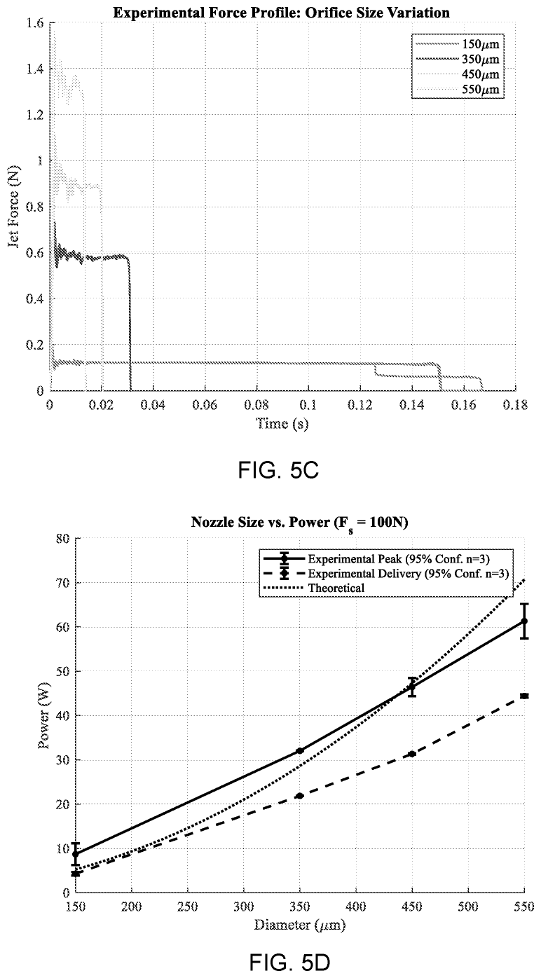

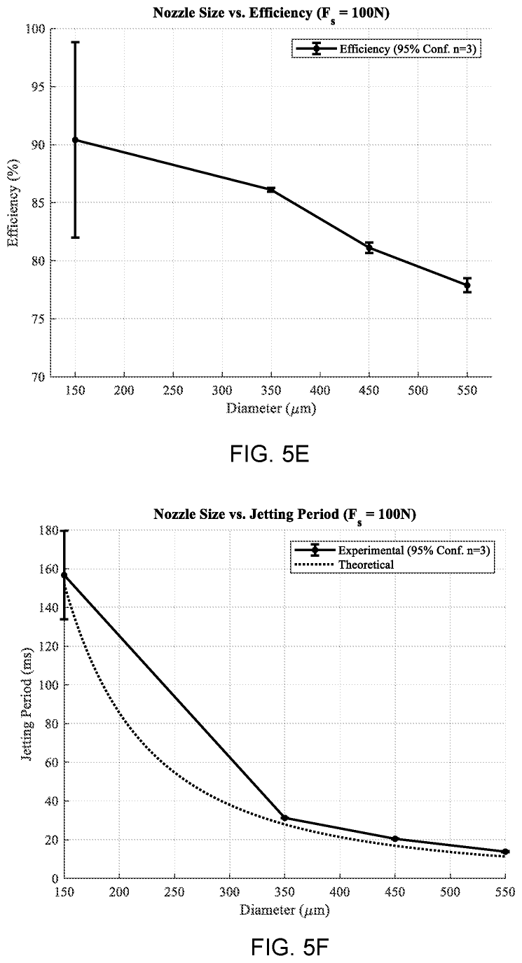

The resulting model was used to determine the jet force and power versus time for different nozzle diameters. The results are shown in FIGS. 5A-5F. The model clearly illustrates how changing the nozzle diameter for a given power system may affect both the peak jet force and jet power as well as the duration of the jet for a set potential energy source such as the assumed linear spring in the model.

In order to validate the above model, a hand-held system and force transducer were used. The test-stand was designed to measure jetting force while varying parameters including nozzle orifice size, initial spring force and final spring force, standoff distance, fluid viscosity, angle of incidence, and expelled volume. The test stand consisted mainly of a hand-held jetting device mounted onto an aluminum rail with sensors for measuring the resulting jetting force. Since the device allowed an operator to quickly switch nozzles and springs if desired it was possible to quickly measure a number of different combinations of jetting parameters. Experiments were performed using a coil spring with an initial spring force of 66 N and a ratio of the final spring force after jetting to the initial spring force of 0.45. A quick-disconnect hose fitting was used as the trigger for the testing rig. A piezoelectric force transducer was used for measuring the thrust from the jet. High-speed video was also used for observing the shape of the jet to verify that the jet was indeed columnar, and not a spray. Five replicates were performed for each experimental data point. An ampule volume of 200 uL of 100% deionized water was used for all experiments except for those in which fluid viscosity was varied.

In each case the nozzle efficiency was deduced by comparison with the theoretical energy input into the jet (i.e. minus piston friction). The resulting nozzle efficiencies used to fit the experimentally measured data varied between about 75% and 85% though the efficiency for the 200 μm nozzle was about 88%, as shown in FIGS. 5C-5F. Accordingly, it may be desirable to determine a nozzle efficiency of an outlet that a jet is emitted from when designing a device for delivering a desired jet power. In either case, the experiments confirmed the ability to predict the jetting power of a device with modeling and experimental determination of appropriate parameters.

### Example: Ex Vivo Testing

Without wishing to be bound by theory, in some embodiments, a gastrointestinal based jetting device can achieve two types of injection: submucosal injection, where a depot is formed directly beneath or within the submucosal tissue, and intramuscular injection, where the jet is deposited into the muscularis. It was also assumed that the power requirements for depot formation in the gastrointestinal tract are lower than that of skin, as mucosal cells are softer than dermal cells, and in most cases, much thinner. To support these assumptions, 200 μL of contrast agent and/or tissue die was injected into 5 cm×5 cm samples of porcine intestinal and gastric tissue. A pneumatic cylinder with a final to initial compression ratio of 0.90 was used for all tests for displacing a piston to expel a jet through outlets of various diameter. The device was mounted vertically and tissue was placed directly beneath it, on top of a saline-soaked sponge in a petri-dish. The tissue was then brought into direct contact with the outlet nozzle using a bench-top scissor jack. Tissue was harvested from lab-raised pigs and tested within six hours of excision. Micro-CT was used to analyze the depot efficiency of delivery for each sample. A suspension of 5% wt. barium sulfate was employed as a contrast agent for injection. Tissue samples were scanned within ten minutes of injection so that diffusion was minimized prior to evaluation.

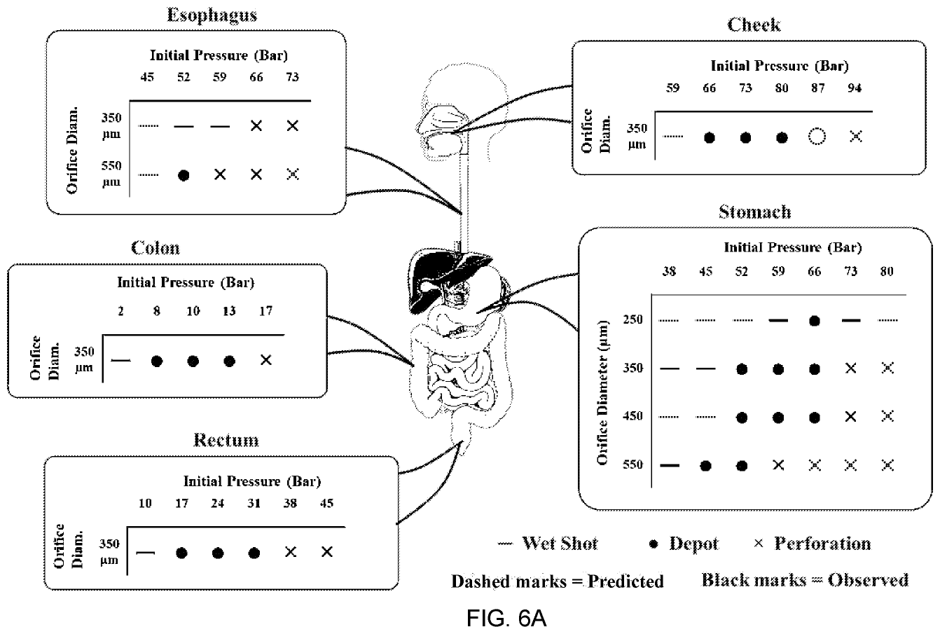

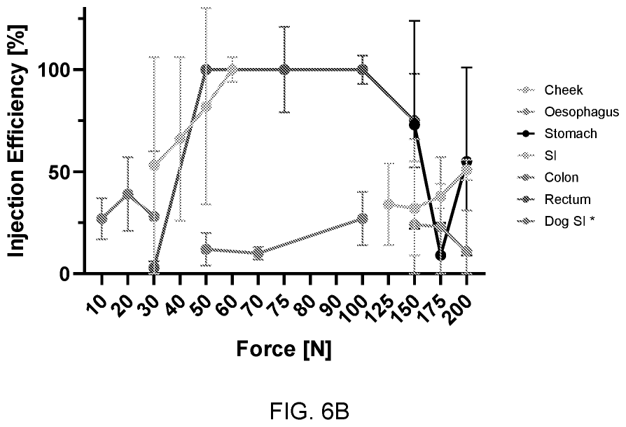

Through application of the experimental and imaging methods mentioned above, jetting performance in the GI tract in different anatomical structures for different initial pressures were determined with ranges corresponding to wet shots where most of the liquid failed to penetrate the tissue, depot formation, and perforation of the tissue respectively. A depot was determined to have been formed when a visible depot was observed both visually and through micro-CT scan. A “perforation” was defined to mean that a clear wound was visible on the serosal side of the tissue and little to no contrast agent was contained within the tissue. FIG. 6A shows the initial pressures and corresponding orifice diameters used to form jets in different tissue including the esophagus, colon, rectum, cheek, and stomach. Wet shots, depot formation, and perforations for the tissue are indicated by dashes, circles, and x's respectively. Additionally, predicted jet performance is indicated by dashed symbols. FIG. 6B shows additional measured data for jet injection efficiency versus jetting force for a variety of tissues, including check, esophagus, stomach, small intestine (SI), colon, rectum, and dog SI.

The experimental data was used to calculate minimum observed peak power for depot formation in each organ based on the measured data. The results are tabulated in Table II. Note that a lower minimum requirement might be possible given smaller nozzle sizes which were not measured. The calculated jet powers were calculated assuming a nozzle efficiency of 80%. As expected, the minimum peak power for depot formation in each tissue type varied widely from organ to organ.

TABLE II

| | Min. peak power |
|---|---|
| Organ | to achieve Depot |
| Cheek | 52 W |
| Esophagus | 90 W |
| Stomach | 9 W |
| Colon | 2 W |
| Rectum | 7 W |

With regards to the stomach, the optimal power to form a depot with high efficiency was approximately 21.4 W. However, depots start forming from about 9 W and perforations was observed with higher jetting efficiencies at approximately 30 W and a 450 μm nozzle diameter. Additionally, perforations were observed starting around 40 W.

Testing was also conducted on small intestine tissue. The range of peak powers at which depots in the intestine were formed ranged between about 3 W and 6.5 W before perforation was observed.

### Example: Depot Efficiency Testing

From the above noted model and experimental data, and without out wishing to be bound by theory, increasing the diameter of an outlet results in higher force, and thus higher peak power. Therefore, it may be desirable to identify the biggest nozzle orifice diameter and the least input force to achieve the highest efficiency of depot formation (volume of loaded drug vs. volume of depot formed). To validate this concept, testing of the efficiency of depot formation was conducted.

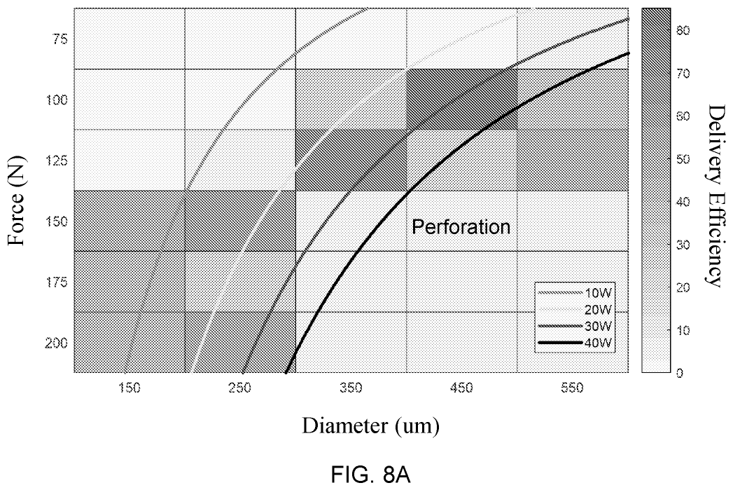

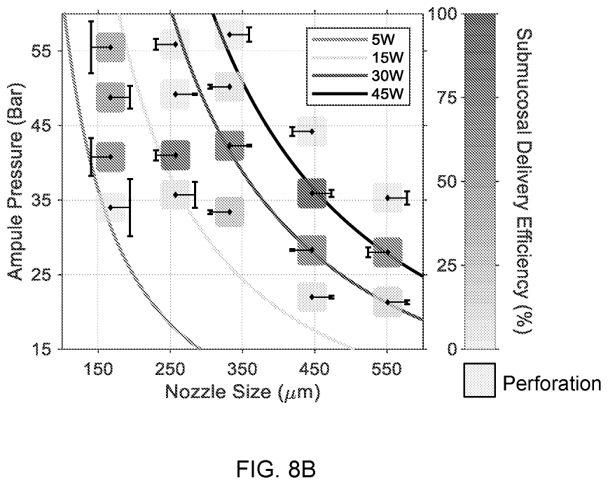

FIGS. 8A-8B depicts an experimental summary of parametric inputs (jetting force or pressure, nozzle diameter, and jetting power) and their resulting delivery efficiencies in stomach tissue. FIG. 8A was plotted using Force (N) and FIG. 8B was plotted using pressure (Bar) applied to the API reservoir. The lines define curves of constant power assuming a piston diameter of 6 mm, density of 1200 kg/m³ and constant system efficiency of 80%. Shaded regions marked as perforated are data points where perforation of tissue was observed. In the chart the actual point at which the data is applicable in each box is the exact center of the box. As shown in FIG. 8, a broad range of jetting forces and pressures combined with varying diameters may result in injection efficiencies greater than 50% for the stomach. For stomach tissue, high efficiencies without perforation was achieved in the different tests with jetting powers between 9 W and 40 W in FIG. 8A as well as between 5 W and 45 W in FIG. 8B for jet diameters between 150 μm and 550 μm. Depot formation of greater was also observed with combinations of jetting forces between 75 N and 200 N as well as jetting pressures between 15 Bar and 60 Bar. In particular, high efficiencies greater than 70% may be achieved for jetting powers between 20 W and 40 W with a jet diameter between 250 μm and 550 μm and a jetting force between or equal to 75 N and 175 N and/or a jetting pressure between or equal to 15 Bar and 45 Bar. It is expected that that more refined combinations of the above-noted ranges are to be identified with further experimental testing. Accordingly, while certain ranges were shown having higher efficiency than other ranges in this particular experiment, additional effective ranges for stomach delivery are expected and the present disclosure is not so limited.

As shown in FIG. 8B, a broad range of jetting pressures and diameters may result in injection efficiencies greater than 50% for the stomach. For stomach tissue, high efficiencies without perforation may be achieved with jetting powers between 5 W and 45 W for jet diameters between 150 μm and 550 μm and jetting pressures between 15 and 60 Bar. In particular, high efficiencies greater than 70% may be achieved for jetting powers between 20 W and 40 W with a jet diameter between 250 μm and 550 μm and a jetting force between 15 and 45 Bar. It is expected that that more refined combinations of the above-noted ranges are to be identified with further experimental testing. Accordingly, while certain ranges were shown having higher efficiency than other ranges in this particular experiment, additional effective ranges for stomach delivery are expected and the present disclosure is not so limited.

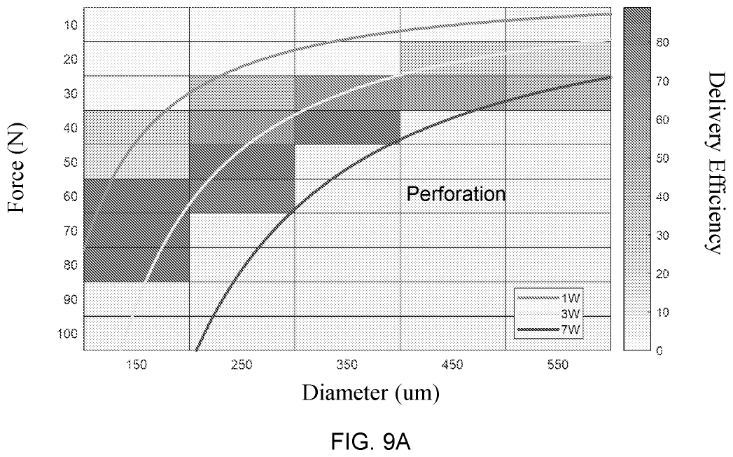

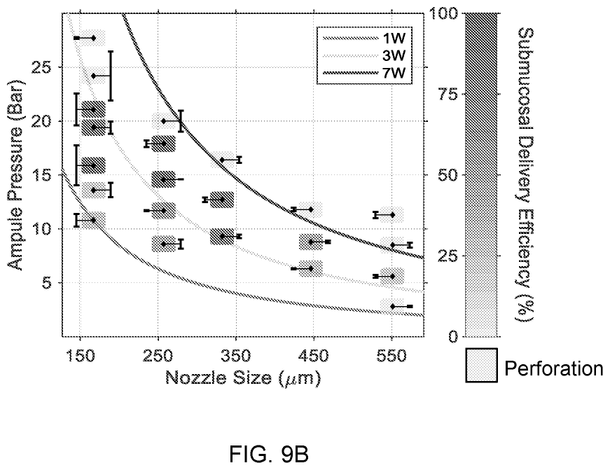

FIG. 9A depicts a preliminary experimental summary of parametric inputs (jetting force or pressure, nozzle diameter, and jetting power) and their resulting delivery efficiencies in intestinal tissue. FIG. 9A was plotted using Force (N) and FIG. 9B was plotted using pressure (Bar) applied to the API reservoir. The lines define curves of constant power assuming a piston diameter of 6 mm, density of 1200 kg/m³ and constant system efficiency of 80%. Shaded regions marked as perforated are data points where perforation of tissue was observed. In the chart the actual point at which the data is applicable in each box is the exact center of the box. As shown in FIG. 9, a broad range of jetting forces and diameters may result in injection efficiencies greater than 50% for intestinal tissue. For intestinal tissue, high efficiencies without perforation may be achieved with jetting powers between 3 W and 6.5 W for jet diameters between 150 μm and 550 μm and jetting forces between 20 and 90 N. In particular, high efficiencies greater than 70% may be achieved for jetting powers between 3 W and 6 W with a jet diameter between 150 μm and 350 μm and a jetting force between 30 and 80 N. It is expected that that more refined combinations of the above-noted ranges are to be identified with further experimental testing. Accordingly, while certain ranges were shown have higher efficiency than other ranges in this particular experiment, additional effective ranges for intestinal tissue delivery are expected and the present disclosure is not so limited.

As shown in FIG. 9B, a broad range of jetting pressures and diameters may result in injection efficiencies greater than 50% for intestinal tissue. For intestinal tissue, high efficiencies without perforation may be achieved with jetting powers between 3 W and 6.5 W for jet diameters between 150 μm and 550 μm and jetting pressures between 5 and 20 Bar. In particular, high efficiencies greater than 70% may be achieved for jetting powers between 3 W and 6 W with a jet diameter between 150 μm and 350 μm and a jetting pressure between 10 and 20 Bar. It is expected that that more refined combinations of the above-noted ranges are to be identified with further experimental testing. Accordingly, while certain ranges were shown have higher efficiency than other ranges in this particular experiment, additional effective ranges for intestinal tissue delivery are expected and the present disclosure is not so limited.

### Example: In Vivo Testing

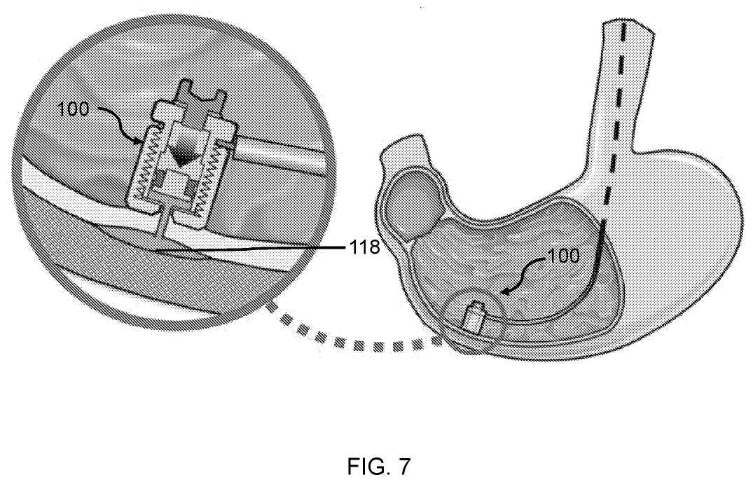

Studies were performed by trained veterinary technicians at MIT's animal testing facilities. Yorkshire pigs weighing between 70 and 90 kg were used. All studies were terminal studies (meaning the animal was euthanized immediately afterward). FIG. 7 illustrates the use of a tethered device 100 which was used to deliver a jet of insulin to form depots 118 in the stomach wall of the animal. The testing protocol is described further below.

During testing, a weight of the pig was determined, and a quantity of insulin was selected to achieve a dose of 0.5 Units per kilogram (1 Unit=0.0347 mg). The powder insulin was then added to a 0.1M NaOH solution, and PF68 and HEPES were used as stabilizing agents. From there 0.1M HCl was added to assist with dissolution of the insulin, and deionized water was added if further dilution was desired. Finally, small amounts of NaOH were added until the pH of the solution reached a value greater than 8.0 (at which insulin is most stable). This formulation procedure was carried out the morning or evening before each in vivo study, and the resulting solution it was stored at 4° C. until the time of administration.

The device was loaded with API and CO2 in the operating room where the animal was sedated and intubated. The device was deployed either by direct placement into the stomach through a laparotomy, or via an over-tube with an endoscope and snare. Of the five deployments performed with this device, the first three were performed by laparotomy, and the latter two by endoscope. Triggering generally occurred within 15 minutes and could be identified through recoil and minor foaming near the base of the device.

Blood samples were collected by ear catheter or femoral catheter. Samples were taken in approximately 15 minute intervals for an hour before scheduled deployment in order to ensure the stability of blood glucose levels. After deployment, blood samples were collected in five-minute intervals for the first 30 minutes, then in 15-minute intervals until two hours after deployment. Samples were stored on ice in 3 mL EDTA tubes until the completion of the study. The blood-glucose level was monitored at each draw using commercial glucose monitoring strips. If the level dropped below 20 mg/dL, an intravenous infusion of 12 mL of 50% dextrose solution was administered to avoid hyperglycemia. The samples were then subsequently analyzed with a custom Ezyme-linked immunosorbent assay (ELISA) for blood glucose levels.

Three of the five device tests produced a drop in blood glucose levels and corresponding increase in blood-plasma insulin concentration. The fact that certain devices delivered insulin and others did not was likely due to manufacturing variation in device orifice size. For the prototype device, orifice sizes were observed to vary widely, a problem which was addressed in later versions through automation of machining. In either case, these tests confirm the feasibility of oral delivery of biologics.

Similar testing was also conducted using a tethered device in the small intestine of a porcine model. Similar results showing the bioavailability of jet delivered insulin were also observed for jet deployed insulin in the small intestine as well.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A drug delivery device configured for administration to a subject, the device comprising:
a reservoir configured to contain an active pharmaceutical ingredient;
a potential energy source;
a trigger operatively associated with the potential energy source, wherein the trigger is configured to actuate within a stomach of the subject; and
an outlet in fluid communication with the reservoir, wherein when the trigger is actuated the potential energy source compresses the reservoir to jet the active pharmaceutical ingredient from the reservoir through the outlet with a velocity sufficient to penetrate a tissue of the stomach adjacent to the outlet, and wherein a peak power provided by the potential energy source to form the jet of the active pharmaceutical ingredient is between 9 Watts (W) and 130 W.

2. A drug delivery device configured for administration to a subject, the device comprising:
a reservoir configured to contain an active pharmaceutical ingredient;
a potential energy source;
a trigger operatively associated with the potential energy source, wherein the trigger is configured to actuate in response to one or more predetermined conditions; and
an outlet in fluid communication with the reservoir, wherein when the trigger is actuated the potential energy source compresses the reservoir to jet the active pharmaceutical ingredient from the reservoir through the outlet with a velocity between 20 m/s and 250 m/s and wherein a peak power provided by the potential energy source to form the jet of the active pharmaceutical ingredient is between 9 Watts (W) and 130 W.

3. The drug delivery device of claim 2 wherein the trigger is configured to actuate within a stomach of the subject.

4. The drug delivery device of claim 2 wherein when the trigger is actuated the potential energy source compresses the reservoir to jet the active pharmaceutical ingredient from the reservoir through the outlet with a velocity sufficient to penetrate a tissue of the stomach adjacent to the outlet.

5. The drug delivery device of claim 1, wherein the peak power is between 9 W and 70 W.

6. The drug delivery device of claim 1, wherein the peak power is between 9 W and 12 W.

7. The drug delivery device of claim 1, wherein the outlet, the reservoir, and the potential energy source are configured to form a depot of the active pharmaceutical ingredient in a tissue of the stomach without perforating a muscularis layer of the stomach.

8. A drug delivery device configured for administration to a subject, the device comprising:
a reservoir configured to contain an active pharmaceutical ingredient;
a potential energy source;
a trigger operatively associated with the potential energy source, wherein the trigger is configured to actuate within a stomach of the subject; and
an outlet in fluid communication with the reservoir, wherein when the trigger is actuated the potential energy source compresses the reservoir to jet the active pharmaceutical ingredient from the reservoir through the outlet with a velocity sufficient to penetrate a tissue of the stomach adjacent to the outlet, and wherein the outlet, the reservoir, and the potential energy source are configured to form a depot of the active pharmaceutical ingredient in a tissue of the stomach without perforating a muscularis layer of the stomach.

9. A drug delivery device configured for administration to a subject, the device comprising:
a reservoir configured to contain an active pharmaceutical ingredient;
a potential energy source;
a trigger operatively associated with the potential energy source, wherein the trigger is configured to actuate in response to one of more predetermined conditions; and
an outlet in fluid communication with the reservoir, wherein when the trigger is actuated the potential energy source compresses the reservoir to jet the active pharmaceutical ingredient from the reservoir through the outlet with a velocity between 20 m/s and 250 m/s, and wherein the outlet, the reservoir, and the potential energy source are configured to form a depot of the active pharmaceutical ingredient in a tissue of the stomach without perforating a muscularis layer of the stomach.

10. The drug delivery device of claim 9 wherein the trigger is configured to actuate within a stomach of the subject.

11. The drug delivery device of claim 9 wherein when the trigger is actuated the potential energy source compresses the reservoir to jet the active pharmaceutical ingredient from the reservoir through the outlet with a velocity sufficient to penetrate a tissue of the stomach adjacent to the outlet.

12. The drug delivery device of claim 8, wherein a peak power provided by the potential energy source to form the jet of the active pharmaceutical ingredient is between 9 W and 130 W.

13. The drug delivery device of claim 1, wherein the outlet, the reservoir, and the potential energy source are configured to form the depot in the submucosal tissue with a depot efficiency of at least 50%.

14. The drug delivery device of claim 1, wherein the velocity of the jet is between 80 m/s and 130 m/s.

15. The drug delivery device of claim 1, wherein a maximum transverse dimension of the outlet is between 50 μm and 450 μm.

16. The drug delivery device of claim 1, wherein the potential energy source comprises at least one of a compressed gas, a spring, an explosive, and a reaction chamber.

17. The drug delivery device of claim 1, wherein an overall volume of the drug delivery device is less than 3000 mm³.

18. The drug delivery device of claim 1, further comprising the active pharmaceutical ingredient disposed in the reservoir.

19. The drug delivery device of claim 1, wherein the subject is a human subject.

20. A method of administering an active pharmaceutical ingredient to a subject, the method comprising:
triggering deployment of a jet of the active pharmaceutical ingredient within a stomach of the subject; and
penetrating a tissue of the stomach of the subject with the jet, wherein a peak power applied to form the jet of the active pharmaceutical ingredient is between 9 W and 130 W.

21. The method of claim 20, wherein the peak power is between 9 W and 70 W.

22. The method of claim 20, wherein the peak power is between 9 W and 12 W.

23. The method of claim 20, further comprising forming a depot of the active pharmaceutical ingredient within the tissue of the stomach without perforating a muscularis layer of the stomach.

24. A method of administering an active pharmaceutical ingredient to a subject, the method comprising:
triggering deployment of a jet of the active pharmaceutical ingredient within a stomach of the subject;
penetrating a tissue of the stomach of the subject with the jet; and
forming a depot of the active pharmaceutical ingredient within the tissue of the stomach without perforating a muscularis layer of the stomach.

25. The method of claim 24, wherein a peak power applied to form the jet of the active pharmaceutical ingredient is between 9 W and 130 W.

26. The method of claim 20, wherein a depot efficiency of the depot formed by the active pharmaceutical ingredient is at least 50%.

27. The method of claim 20, wherein a velocity of the jet is between 80 m/s and 130 m/s.

28. The method of claim 20, wherein the jet has a maximum transverse dimension between 50 μm and 450 μm.

29. The method of claim 20, further comprising positioning a drug delivery device including the active pharmaceutical ingredient in the stomach of the subject.

30. The method of claim 29, wherein an overall volume of the drug delivery device is less than 3000 mm³.

31. The method of claim 20, wherein the subject is a human subject.

32. A drug delivery device configured for administration to a subject, the device comprising:
a reservoir configured to contain an active pharmaceutical ingredient;
a potential energy source;
a trigger operatively associated with the potential energy source, wherein the trigger is configured to actuate within a small intestine of the subject; and
an outlet in fluid communication with the reservoir, wherein when the trigger is actuated the potential energy source compresses the reservoir to jet the active pharmaceutical ingredient from the reservoir through the outlet with a velocity sufficient to penetrate a tissue of the small intestine adjacent to the outlet, and wherein a peak power provided by the potential energy source to form the jet of the active pharmaceutical ingredient is between 3 W and 6.5 W.

33. A drug delivery device configured for administration to a subject, the device comprising:
a reservoir configured to contain an active pharmaceutical ingredient;
a potential energy source;
a trigger operatively associated with the potential energy source, wherein the trigger is configured to actuate in response to one or more predetermined conditions; and
an outlet in fluid communication with the reservoir, wherein when the trigger is actuated the potential energy source compresses the reservoir to jet the active pharmaceutical ingredient from the reservoir through the outlet with a velocity between 40 m/s and 80 m/s, and wherein a peak power provided by the potential energy source to form the jet of the active pharmaceutical ingredient is between 3 W and 6.5 W.

34. The drug delivery device of claim 33 wherein the trigger is configured to actuate within a small intestine of the subject.

35. The drug delivery device of claim 33 wherein when the trigger is actuated the potential energy source compresses the reservoir to jet the active pharmaceutical ingredient from the reservoir through the outlet with a velocity sufficient to penetrate a tissue of the small intestine adjacent to the outlet.

36. The drug delivery device of claim 32, wherein the outlet, the reservoir, and the potential energy source are configured to form a depot of the active pharmaceutical ingredient in a tissue of the small intestine without perforating a muscularis layer of the small intestine.

37. A drug delivery device configured for administration to a subject, the device comprising:
a reservoir configured to contain an active pharmaceutical ingredient;
a potential energy source;
a trigger operatively associated with the potential energy source, wherein the trigger is configured to actuate within a small intestine of the subject; and
an outlet in fluid communication with the reservoir, wherein when the trigger is actuated the potential energy source compresses the reservoir to jet the active pharmaceutical ingredient from the reservoir through the outlet with a velocity sufficient to penetrate a tissue of the small intestine adjacent to the outlet, and wherein the outlet, the reservoir, and the potential energy source are configured to form a depot of the active pharmaceutical ingredient in a tissue of the small intestine without perforating a muscularis layer of the small intestine.

38. A drug delivery device configured for administration to a subject, the device comprising:
a reservoir configured to contain an active pharmaceutical ingredient;
a potential energy source;
a trigger operatively associated with the potential energy source, wherein the trigger is configured to actuate in response to one or more predetermined conditions; and
an outlet in fluid communication with the reservoir, wherein when the trigger is actuated the potential energy source compresses the reservoir to jet the active pharmaceutical ingredient from the reservoir through the outlet with a velocity between 40 m/s and 80 m/s adjacent to the outlet, and wherein the outlet, the reservoir, and the potential energy source are configured to form a depot of the active pharmaceutical ingredient in a tissue of the small intestine without perforating a muscularis layer of the small intestine.

39. The drug delivery device of claim 38 wherein the trigger is configured to actuate within a small intestine of the subject.

40. The drug delivery device of claim 38 wherein when the trigger is actuated the potential energy source compresses the reservoir to jet the active pharmaceutical ingredient from the reservoir through the outlet with a velocity sufficient to penetrate a tissue of the small intestine adjacent to the outlet.

41. The drug delivery device of claim 37, wherein a p leak power provided by the potential energy source to form the jet of the active pharmaceutical ingredient is between 3 W and 6.5 W.

42. The drug delivery device of claim 32, wherein the outlet, the reservoir, and the potential energy source are configured form the depot in the submucosal tissue with a depot efficiency of at least 50%.

43. The drug delivery device of claim 32, wherein the velocity of the jet is between 40 m/s and 80 m/s.

44. The drug delivery device of claim 32, wherein a maximum transverse dimension of the outlet is between 50 μm and 450 μm.

45. The drug delivery device of claim 32, wherein the potential energy source comprises at least one of a compressed gas, a spring, an explosive, and a reaction chamber.

46. The drug delivery device of claim 32, wherein an overall volume of the drug delivery device is less than 3000 mm³.

47. The drug delivery device of claim 32, further comprising the active pharmaceutical ingredient disposed in the reservoir.

48. The drug delivery device of claim 32, wherein the subject is a human subject.

49. A method of administering an active pharmaceutical ingredient to a subject, the method comprising:
triggering deployment of a jet of the active pharmaceutical ingredient within a small intestine of the subject; and
penetrating a tissue of the small intestine of the subject with the jet, wherein a peak power applied to form the jet of the active pharmaceutical ingredient is between 3 W and 6.5 W.

50. The method of claim 49, further comprising forming a depot of the active pharmaceutical ingredient within the tissue of the small intestine without perforating a muscularis layer of the small intestine.

51. A method of administering an active pharmaceutical ingredient to a subject, the method comprising:
triggering deployment of a jet of the active pharmaceutical ingredient within a small intestine of the subject;
penetrating a tissue of the small intestine of the subject with the jet; and
forming a depot of the active pharmaceutical ingredient within the tissue of the small intestine without perforating a muscularis layer of the small intestine.

52. The method of claim 51, wherein a peak power applied to form the jet of the active pharmaceutical ingredient is between 3 W and 6.5 W.

53. The method of claim 49, wherein a depot efficiency of the depot formed by the active pharmaceutical ingredient is at least 50%.

54. The method of claim 49, wherein a velocity of the jet is between 80 m/s and 130 m/s.

55. The method of claim 49, wherein the jet has a maximum transverse dimension between 50 μm and 450 μm.

56. The method of claim 49, further comprising positioning a drug delivery device including the active pharmaceutical ingredient in the small intestine of the subject.

57. The method of claim 56, wherein an overall volume of the drug delivery device is less than 3000 mm³.

58. The method of claim 49, wherein the subject is a human subject.
